(19) United States
(12) Patent Application Publication
(10) Pub. No.: US 2013/0045991 A1
(43) Pub. Date: Feb. 21, 2013

(54) NEUROPROTECTIVE MODULATION OF NMDA RECEPTOR SUBTYPE ACTIVITIES

(71) Applicant: THE UNIVERSITY OF BRITISH COLUMBIA, VANCOUVER (CA)

(72) Inventors: YU TIAN WANG, VANCOUVER (CA); YITAO LIU, VANCOUVER (CA); TAK PAN WONG, VANCOUVER (CA); LIDONG LIU, VANCOUVER (CA)

(73) Assignee: THE UNIVERSITY OF BRITISH COLUMBIA, VANCOUVER (CA)

(21) Appl. No.: 13/649,592

(22) Filed: Oct. 11, 2012

Related U.S. Application Data
(63) Continuation of application No. 11/995,494, filed on Jun. 4, 2009, now abandoned.
(60) Provisional application No. 60/698,954, filed on Jul. 14, 2005.

(51) Int. Cl. A61K 31/445; A61P 25/00; A61P 25/28; A61P 31/00; A61P 25/08; A61P 9/00; A61P 9/10; A61P 7/04; C12N 5/0793; A61P 25/16

(52) U.S. Cl. 514/317; 435/375

(57) ABSTRACT

In various aspects, the invention provides methods and compositions for modulating NMDA receptor subtype activity, to enhance NR2A-containing NMDA receptor activity relative to NR2B-containing NMDA receptor activity, so as to effect a neuroprotective reduction in excitotoxic NMDA receptor activity

### FIELD

The invention is in the field of pharmacological treatments for conditions affecting neurons.

### BACKGROUND

Synaptic transmission is the process by which neurons communicate by excitatory (generation of an action potential) or inhibitory (inhibition of an action potential following excitation) mechanisms. Excitatory synaptic transmission often occurs by means of the neurotransmitter L-glutamate and its cognate glutamate receptors. Glutamate receptors are the primary excitatory neurotransmitters in the mammalian brain, and are activated in a variety of neurophysiological processes involved in both normal function and disease states. The excessive stimulation of post-synaptic neurons (a phenomenon known as “excitotoxicity”), can lead to neuronal death or apoptosis, and has been implicated in a variety of central nervous system (CNS) disorders.

Classification of glutamate receptors is based on their response to specific agonists such as alpha-amino-3-hydroxy-5-methyl-4-isoxazole propionate (AMPA), N-methyl-Daspartate (NMDA), quisqualic acid (QUIS), kainite (KA), and 2-amino-4-phosphonobutyrate (AP4). NMDA and AMPA receptors are the best known of the glutamate receptors (Dingledine et al., 1999). NMDA receptors are multimeric calcium channels found in several classes of neurons.

Activation of the NMDA receptor may induce programmed cell death (apoptosis) in neurons, and may underlie the loss of neurons and neuronal function in central nervous system disorders ranging from acute brain trauma and stroke to neurodegenerative diseases such as Huntington's, Alzheimer's, and Parkinson's Diseases (Mattson, 2000; Graham et al., 2001; Yu et al., 2001; Nicotera et al., 1999; Hardingham et al., 2002).

NMDA receptors are believed to be tetrameric protein complexes comprised of NR1 subunits with at least one type of NR2 subunit. The NR2B and NR2A subunits are thought to be involved in glutamate binding by NMDA receptors, while the NR1 subunit is thought to be involved in binding the co-agonist glycine. Different NR2 subunits confer distinct electrophysiological and pharmacological properties on the receptors and couple them with different signaling machineries. For instance, it has been suggested that NR2A- and NR2B-containing NMDA receptor subtypes have opposing roles in dictating the direction of synaptic plasticity (Kirson et al., 1996; Tovar et al., 1999; Sheng et al., 1994; Liu et al., 2004). It has been demonstrated using heteromeric NMDA receptors expressed in Xenopus oocytes that oocyte-expressed NR1/NR2A receptors display a higher affinity for certain antagonists and a slightly lower affinity for selected agonists than NR1/NR2B receptors (Buller et al., 1994). The distribution of NR2A mRNA has been correlated with the distribution of “antagonist-preferring” NMDA receptors, defined by high-affinity 3H-2-carboxypiperazine-4-yl-propyl-1-phosphonic (3H-CPP) binding sites. Accordingly, there is evidence that NMDA receptor antagonists may preferentially target NR2A-containing NMDA receptors. Interestingly, NR2A and NR2B are reportedly the predominant NR2 subunits in the adult forebrain, where stroke most frequently occurs.

Neuronal apoptosis induced by activation of the NMDA receptor is thought to be central to the loss of neurons and neuronal function that accompanies stroke, brain trauma and neurodegenerative disorders. The effects of NMDA receptor antagonism illustrates two apparently paradoxical roles: both neuronal apoptosis in developmental models and neuroprotection against ischemic brain damage in stroke models (Hardingham et al., 2002; Ikonomidou et al., 1999; Lee et al., 1999; Arundine et al., 2004). A variety of NMDA antagonists, such as ifenprodil and eliprodil, are thought to have neuroprotective effects. Ro 63-1908, a NMDA ligand having 20,000-fold selectivity for the NR1C and NR2B receptors over NR1C+NR2A receptors, reportedly has a dose-related neuroprotective effect against cortical damage in a model of permanent focal ischemia (Gill et al., 2002).

Molecular and experimental animal studies have consistently demonstrated that over activation of the N-methyl-D-aspartate (NMDA) subtype glutamate receptors is the primary step leading to neuronal injury following insults of stroke and brain trauma (Lee et al., 1999; Arundine et al., 2004; Mattson, 1997; Lipton et al., 1994). Nevertheless, several large scale clinical trials have failed to find the expected efficacy of NMDA receptor antagonists in reducing brain injuries (Lee et al., 1999; Kemp et al., 2002; Ikonomidou et al., 2002). The clinical efficacy of NMDA antagonists remains in question (Hoyte et al., 2004; Roesler, et al. 2003).

There is an ongoing interest in the delineation of pharmacological properties of NMDA receptors that may serve as the basis for more effective therapeutic approaches to a variety of diseases (Kemp et al., 2002; Danton et al., 2004; Krystal et al., 1999). For example, subunit-specific amino acid residues have been identified in the NMDA receptor glutamate-binding pocket (Kinarsky et al., 2005; Blaise et al., 2004; Klein et al., 2001). Similarly, detailed information is available on the NMDA receptor glycine binding site (Foucaud et al., 2003).

It is known that NR2A and NR2B subunits have pharmacologically distinct competitive antagonist binding sites (Christie et al, 2000; Blanchet et al., 1999; Priestley et al., 1995).

There has been a significant degree of interest in the clinical relevance of NR2B selective antagonists (McCauley, 2005; WO2005080317). NR2B selective antagonists (such as CP-101,606; CI-1041; Co-101,244, RG-13579 and RG-1103) have shown promise in some neuroprotective treatments (Nagy et al., 2004). A significant number of NR2B-selective antagonists have been identified (Donevan et al., 2000; White et al, 2000). For example, felbamate, an anticonvulsant used in the treatment of seizures, has been characterized as an NR2B-selective antagonist (Kleckner et al., 1999). A family of structurally related sigma site ligands ligands [eliprodil, haloperidol, ifenprodil, 4-phenyl-1-(4-phenylbutyl)-piperidine and trifluperidol] have been identified as strongly selective antagonists for NR1a/2B receptors (Whittemore et al., 1997). CP101,606, an ifenprodil analog, has been identified as an NMDA receptor antagonist with preference for the NR1/NR2B subunit combination (Brimecombe et al., 1998). A wide variety of NR2B-containing NMDA receptor antagonists have reportedly been the subject of clinical testing, for a wide variety of indications: EVT-101, EVT-103 and EVT-102 (Evotec) for Alzheimer's and Parkinson's diseases and neuropathic pain; RGH-896 (Gedeon Richter) for neuropathic pain and other CNS indications; ED-1529 (Sosei) for neuropathic pain and other pain indications; HON-0001 (Taisho) for neuropathic and other pain conditions; Traxoprodil mesylate (Pfizer) for analgesia and stroke; Ifenprodil (Sanofi-Aventis) for peripheral neuropathies and CNS neurodegenerative disorders (EP698391).

The pharmacology of a catalogue of NR2B-containing NMDA receptor antagonists is relatively well characterized:
i) Ro 25-6981 hydrochloride ([R-(R*,S*)]-α-(4-Hydroxyphenyl)-β-methyl-4-(phenylmethyl)-1-piperidinepropanol; Mutel et al., 1998; Lozovaya et al., 2004).
ii) Ro 64-1908 (1-[2-(4-hydroxy-phenoxy)-ethyl]-4-(4-methyl-benzyl)-piperidin-4-ol; Gill et al., 2002).
iii) Conantokin G (isolated from the venom of the marine cone snail, Conus geographus, also known as [Glu^{3,4,7,10,14}]-Conantokin; Hammerland et al., 1992; Donevan et al., 2000; Williams et al., 2002).
iv) Conantokin R (isolated from the venom of the fish-hunting snail, Conus radiatus; White et al., 2000).
v) Felbamate (Harty et al., 2000).
vi) CP-101,606 (Di et al., 1997; Chazot, 2000; Boyce-Rustay et al., 2004).
vii) Ifenprodil (α-(4-Hydroxyphenyl)-β-methyl-4-benzyl-1-piperidineethanol tartrate salt; Chenard et al.,1999; Gallagher et al., 1996).
viii) HON0001 (Suetake-Koga et al., 2006).
ix) Pentamidine isethionate (Williams et al., 2003).
x) Ro 8-4304 (Kew et al., 1998).
xi) Eliprodil (Avenet et al., 1997).
xii) (3R,4S)-3-[4-(4-fluorophenyl)-4-hydroxypiperidin-1-yl]chroman-4,7-diol (an analogue of CP-101,606; Butler et al., 1998).
xiii) 1-Benzyloxy-4,5-dihydro-1H-imidazol-2-yl-amine (Alanine et al., 2003).
xiv) CI-1041 (Nagy et al., 2004).
xv) Co-101,244 (Nagy et al., 2004).
xvi) RG-13579 (Nagy et al., 2004).
xvii) RG-1103 (Nagy et al., 2004).
xviii) CGX-1007 (a shortened polypeptide based on Conantokin et al., 2006).
xix) CR 3394 (Losi et al., 2006).
xx) (E)-N-(2-[11C]methoxybenzyl)-3-phenyl-acrylamidine (Losi et al., 2006).

NMDA receptor agonists, particularly those that target the NMDAR-associated glycine binding site, are reported to be effective for the treatment of movement disorders such as Parkinsons disease (U.S. Patent Publication US2004/157926 and U.S. Pat. No. 6,228,875). NR2B-selective antagonists of glycine binding are known, such as CGP 61594 (Honer et al., 1998). In contrast, it has been reported that glycine and serine are associated with enhancement of ischemia induced damage (Delkara et al., 1990). In keeping with this putative pathological roll for glycine, it has also been suggested that glycine antagonists are useful in providing neuroprotection against acute insults, Ischemia and stroke (Danysz et al., 1998). Glycine agonists and partial agonists are identified in that paper as follows:

A structurally diverse array of NMDA receptor glycine agonists are known, such as:

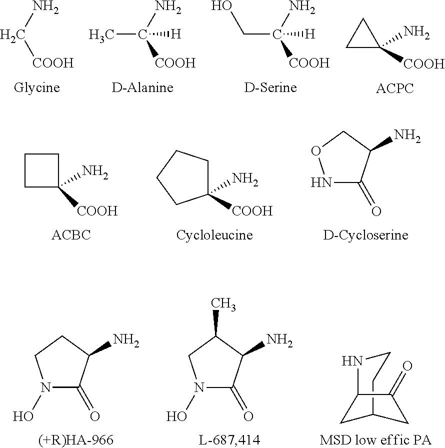

NMDA receptor glycine agonists have been the subject of extensive clinical testing: Nebostinel (Rottapharm) as an antidepressive, antipsychotic, and for cognition disorders (AD, depression, schizophrenia), and age-associated memory impairment; NT-13 (Nyxis Neurotherapies) for neuropathic pain, prevention of stroke and for cognition enhancement; SC-49088 (Pfizer) for Alzheimer's disease and age-associated memory impairment.

The pharmacology of a catalogue of NMDA glycine receptor agonists is relatively well characterized:
i) D-cycloserine ((R)-4-Amino-3-isoxazolidone; Hood et al., 1989; Watson et al., 1990; Singh et al., 1990).
ii) 1-Aminocyclopropanecarboxylic acid and 1-Aminocyclopropanecarboxylic acid hydrochloride (Sheinin et al., 2002; Boje et al., 1998).
iii) CR 2249 ((S)-4-Amino-5[(4,4-dimethylcyclohexyl)amino]-5-oxopentanoic acid, or neboglamine; Lanza et al., 1997).
iv) Glycine (Mayer et al., 1989; Priestley et al., 1995).
v) D-serine (Reggiani et al., 1989).
vi) L-687414 (R(+)-cis-beta-methyl-3-amino-1-hydroxypyrrolid-2-one; Tricklebank et al., 1994; Priestley et al., 1995).
vii) (+)-HA 966 (Millan et al.,1993).
viii) DL-(tetraziol-5-yl)glycine (Schoepp et al., 1994).

Glycine antagonists have proven ineffective in clinical treatments for stroke (Lees et al., 2000; Sacco et al., 2001). In contrast, glycine has reportedly been effective in the treatment of stroke. For example, sublingual application of 1.0-2.0 g/day glycine started within 6 h after the onset of acute ischaemic stroke in the carotid artery territory is reported to exert favourable clinical effects (Guseva et al., 2000).

An alternative approach to implementing glycine-mediated NMDA receptor agonism is to increase extracellular levels of glycine, for example by blocking glycine re-uptake. This may for example be accomplished by blocking glycine re-uptake into neurons through the glyT-1 transporter, for example using drugs such as ALX5407 ((R)-NFPS, R—N-(3-[40-fluorophenyl]-3-[40-phenylphenoxy]propyl)sarcosine), NFPS (N-(3-[40-fluorophenyl]-3-[40-phenylphenoxy]propyl)sarcosine), NPTS (N-(3-phenyl-3-[40-{4-toluoyl}phenoxy]propyl)sarcosine) or ORG24598 (R-(−)-N-[3-[(4-triflouromethyl)phenoxy]-3-phenylpropylglycine). Alternatively, an increase in extracellular levels of D-serine, an alternative glycine site agonist, may be mediated by inhibiting re-uptake of D-serine into glia (Kemp et al., 2002).

Sulphated steroids, such as pregnenolone sulfate, have been shown to potentiate NMDA receptors, including recombinant NR1/NR2A receptors, through binding at sites distinct from the glycine or glutamate binding sites (Park-Chung et al.,1997; Yaghoubi et al., 1998). The toxicity-inducing and -potentiating effects of neurosteroid potentiators of NMDA receptors were may be blocked by NMDA antagonists, such as 4-(3-phosphonopropyl)2-piperazinecarboxylic acid (CPP) and MK-801 (Guarneri et al., 1998). The action of PS is reportedly larger on NR1a/NR2A than on NR1a/NR2B channels (Ceccona et al., 2001). Pregnenolone sulfate (PS) reportedly enhances the efficacy of glutamate and glycine as NR1/NR2A receptor agonists (Malayev et al., 2002). The therapeutic potential of steroids in treating conditions of the CNS has been recognized (Hamilton, 2001).

A variety of compounds have been identified as partial agonists, antagonists, and inverse agonists at the polyamine recognition site on NMDA receptors (Williams et al., 1991; Rock and Macdonald, 1995). Well tolerated polyamine NMDA antagonists have been identified, such as memantine (1-amino-3,5-dimethyl-adamantane; Parsons et al.,1999).

A wide variety of methods are known for identifying additional compounds that modulate the activity of NMDA receptors (U.S. Pat. Nos. 5,849,895; 5,985,586; 6,956,102; 6,521,413; 6,316,611; 6,111,091; 6,376,660; 6,469,142; 6,864,358; 6,825,322; 6,033,865). Methods are also know for identifying excitatory glycine receptor ligands (U.S. Patent Publications 2003/92004 and US 2004/33500).

### SUMMARY

In alternative aspects, the invention provides methods and compounds for modulating NMDA receptor subtype activity. For example, NMDA receptor activity may be modulated in a neuron having NR2A-containing NMDA receptors and NR2B-containing NMDA receptors. This may for example involve treating a subject with one or more NMDA receptor modulating compounds in an amount that is effective to enhance NR2A-containing NMDA receptor activity, relative to NR2B-containing NMDA receptor activity. In this way, the invention may be used to effect a neuroprotective reduction in excitotoxic NMDA receptor activity, for example to treat a neurodegenerative condition such as an acute ischemic episode. The NMDA receptor modulating compounds may include an NMDA receptor agonist and an NMDA receptor antagonist, which may for example be used in combination. The NMDA receptor antagonist may for example be an NR2B-containing NMDA receptor selective antagonist.

NMDA receptor agonists and antagonist for use in various aspects of the invention may for example be selected from the group consisting of: NMDA receptor glutamate binding site antagonists; NMDA receptor glycine binding site agonists or antagonists; NMDA receptor polyamine binding site agonists or antagonists; and, NMDA receptor steroid binding site agonists or antagonists. Such compounds may for example be selected from compounds listed herein, such as those identified in the Background, compounds identified in references cited herein, or other compounds having the requisite activity.

In various aspects, the invention involves the use of agonists of an NR2A-containing NMDA receptor. For example, a pharmacologically effective amount of an agonist of an NR2A-containing NMDA receptor may be administered to modulate neuronal survival or death. Neuronal survival or death may also be modulated by administration of an NR2B-containing NMDA receptor antagonist in combination with an agonist of an NR2A-containing NMDA receptor. The agonist of an NR2A-containing NMDA receptor may for example be an NMDA receptor glycine site agonist.

In accordance with another aspect of the invention there is provided the use of an NR2A-containing NMDA receptor agonist to formulate a medicament for use to modulate neuronal cell death or have an anti-apoptotic effect in an animal, such as a human subject. In accordance with another aspect of the invention, there is provided a method of identifying an agonist of an NR2A-containing NMDA receptor, the method comprising exposing a neuronal cell to an apoptosis-inducing insult and to a candidate chemical entity, and assaying for apoptosis. According to another aspect of the invention, there is provided medicaments comprising NR2A-containing NMDA receptor agonists. In one embodiment, such medicaments include an NR2A-containing NMDA agonist in a pharmacologically effective amount sufficient to reduce or substantially inhibit neuronal cell death, and a pharmaceutically acceptable excipient.

In alternative embodiments, the invention provides: methods of modulating neuronal survival by administering a pharmacologically effective amount of an agonist of an NR2A-containing NMDA receptor; methods of modulating neuronal death by administering a pharmacologically effective amount of an agonist of an NR2A-containing NMDA receptor; methods of modulating neuronal death by administering a pharmacologically effective amount of an NR2B-containing NMDA receptor antagonist in combination with an NMDA receptor glycine site agonist; methods of modulating neuronal survival by administering a pharmacologically effective amount of an NR2B-containing NMDA receptor antagonist in combination with an NMDA receptor glycine site agonist; use of an NR2A-containing NMDA receptor agonist to formulate a medicament for use to treat an acute brain injury or neurodegenerative disorder in a human; use of an NR2A-containing NMDA receptor agonist to formulate a medicament for use to modulate neuronal cell death in an animal; use of an NR2A-containing NMDA receptor agonist to formulate a medicament for use to have an anti-apoptotic effect on neuronal cells in an animal; use of an NR2A-containing NMDA receptor agonist to formulate a medicament for use to have a cell-survival promoting effect on neuronal cells in an animal; or, methods of identifying an agonist of an NR2A-containing NMDA receptor, the methods comprising exposing a neuronal cell to an apoptosis-inducing insult and to a candidate chemical entity, and assaying for apoptosis.

In alternative embodiments, neuronal death amenable to treatments in accordance with the invention may, for example, result from an acute brain injury such as stroke, trauma or oxygen deprivation, or may result from or cause a neurodegenerative disorder such as Huntington's Disease, Alzheimer's Disease or amyotrophic lateral sclerosis.

### BRIEF DESCRIPTION OF THE DRAWINGS

FIG. 1. Functional NR2A and NR2B-containing NMDA receptors are present in cultured neurons and are preferentially blocked by their respective antagonists. Whole cell recording was performed at a holding membrane potential of −60 mV in an extracellular solution supplemented with 10 μM CNQX, 0.5 μM TTX, 10 μM bicuculline. A. example traces of whole-cell currents evoked by a brief perfusion of 50 μM NMDA (plus 10 μM glycine and 5 μM strychnine) from a multi-barrel fast perfusion system in the absence or presence of specific NR2A-(NVP-AAM077, 0.4 μM), or NR2B-antagonists (Ro25-6981, 0.5 μM) or both. The percentage blockade of the NMDA-induced currents by sequential application of these two antagonists is summarized in histogram in B. Pre-application of Ro25-6981 did not alter the percentage blockade produced by NVP-AAM077 (p=0.97; in the absence vs in the presence of Ro25-6981), whereas pre-treatment of NVP-AAM077 produced a small, albeit statistically non-significant, reduction in the percentage blockade of the currents produced by Ro25-6981 (5.6%; p=0.19).

FIG. 2. NR2A- and NR2B-containing NMDA receptors exert opposing effects on NMDA-induced excitotoxic neuronal damage. Cortical neuronal cultures were treated without (control) or with NMDA (50 μM plus 10 μM glycine) for 20 min, and examined for neuronal cell death after 20 h. A. Representative images of Hoechst-33342 stained neurons illustrate the differential effects of co-application of NMDA with NR2A antagonist NVP-AAM077 (NVP; 0.4 μM) or NR2B antagonist Ro 25-6981 (Ro; 0.5 μM) on 25 NMDA-induced neuronal damage. NMDA stimulation produced neuronal damage such as chromatin condensation and/or fragmentation which were aggravated in the presence of NVP-AAM077, but eliminated in the presence of Ro 25-6981. B. Cell death ELISA assay for apoptosis quantifies the differential effects of NVP-AAM077 and Ro 25-6981 on NMDA-induced neuronal apoptosis. Data are presented as the difference in apoptosis levels as a percentage of control. ** denotes p<0.001 compared with non-treated control; # and ## denote p<0.05 and p<0.001, respectively, compared with NMDA treatment alone; n=18 tissue culture wells from three separate experiments for each group. C. NR2A- and NR2B-containing receptors have opposing actions on cell survival (Akt activation) and death (caspase-3 activation) signaling pathways. Upper panels: Cell lysates from cultured neurons treated as indicated were sequentially immunoblotted with antibodies specific to Akt phosphorylated on serine 473, the active form of the enzyme (p-Akt), and total Akt (Akt). Lower panels: Cell lysates were sequentially immunoblotted with antibodies that specifically recognize cleaved caspase-3 (activated caspase-3; casp3) and beta-tubulin (Tubulin).

FIG. 3. Activation of synaptic NR2B-containing NMDA receptors produces a pro-apoptotic action which is masked by a predominant synaptic NR2A-containing receptor-mediated cell survival promoting effect. A. Functional synaptic NR2B-containing receptors are present in cortical neurons in culture. Spontaneous miniature excitatory postsynaptic currents (mEPSCs) were recorded in whole-cell voltage-clamp mode at a holding membrane potential of −60 mV in the presence of tetrodotoxin (0.5 μM) and bicuculline (10 μM) with zero added Mg2+. Aa. Examples of mEPSC traces (averaged from 100 individual events) obtained in the absence (Control) and presence of Ro 25-6981 (Ro; 0.5 μM) or the broad spectrum NMDA receptor antagonist APV (APV; 50 μμM). Ab. Total NMDA receptor-mediated component of mEPSCs was obtained by subtracting the averaged mEPSC recorded in the presence of APV from the averaged control mEPSC and expressed as charge transfer (Control-APV; shaded area). Ac. The NR2B-containing receptor component was obtained by subtracting the averaged mEPSC recorded in the presence of Ro 25-6981 (Ro) from the averaged control mEPSC (Control-Ro; shaded area). Ad. Bar graph summarizes data obtained from five individual neurons. Charge transfer is equivalent to the area of the shaded regions. B. Enhanced activation of synaptic NR2A- and NR2B-containing receptors exerts opposing actions on neuronal survival and death. Potentiation of synaptic NMDA receptor activation was achieved by increasing the presynaptic release of glutamate by incubating cultured neurons with bicuculline (Bic; 50 μM) for 4 h in the absence or presence of NR2-containing receptor specific antagonists. Blockade of NR2A- (Bic+NVP), but not NR2B- (Bic+Ro), containing NMDA receptors increased neuronal apoptosis. The NR2A blockade-induced apoptosis was prevented by a further blockade of NR2B-containing receptors (Bic+NVP+Ro). C. Spontaneously activated synaptic NR2A- and NR2B-containing receptors also have opposing roles in promoting neuronal survival and death. Incubation of neurons with NVP-AAM077 (NVP), but not Ro 25-6981 (Ro), for an extended duration (48 h) in the absence of bicuculline stimulation was sufficient to produce an increase in apoptosis. The NVP-AAM077-induced apoptosis was prevented by addition of Ro 25-6981 (NVP+Ro). Thus, both synaptic NR2A- and NR2B-containing subpopulations of NMDA receptors are spontaneously activated by presynaptically released glutamate, exerting counteracting effects on cell survival and death, but synaptic NR2A-containing receptor activation is predominant and required for maintaining normal neuronal survival. ** p<0.001 compared with control; n=16 in (B) and 10-12 in (C) for each group from three separate experiments.

FIG. 4. Activation of extrasynaptic NR2A-containing NMDA receptors promotes cell survival, protecting against extrasynaptic NR2B-containing receptor-mediated and non-NMDA receptor-dependent neuronal death. A. Functional NR2A-containing NMDA receptors are present at extrasynaptic sites. Whole-cell recordings were performed at a holding membrane potential of −60 mV. Aa. Averaged traces of mEPSCs showing an APV-sensitive (50 μM) NMDA receptor-mediated component (Control-APV). Ab. Averaged traces of mEPSCs showing the blockade of synaptic NMDA receptors by the open channel blocker MK-801 (10 μM plus 50 μM bicuculline, 10 min), as demonstrated by the elimination of the NMDA receptor-mediated component of the mEPSCs (Control-APV). Ac. Example traces of whole-cell currents evoked by NMDA (200 μM) following the blockade of synaptic NMDA receptors with MK-801 in the absence (A; control) or presence of Ro 25-6981 (B; 0.5 μM) or Ro 25-6981 plus NVP-AAM077 (C; 0.4 μM). NMDA receptor-mediated currents were evoked by fast application of NMDA within 10 min of washing out MK-801 and bicuculline. Currents remaining following the blockade of extrasynaptic NR2B-containing receptors were virtually abolished by the addition of NVP-AAM077, suggesting the presence of functional extrasynaptic NR2A-containing NMDA receptors in these neurons. Ad. Histogram summarizes data from 5 individual neurons. B. Activation of extrasynaptic NR2A-containing NMDA receptors protects against neuronal death mediated by extrasynaptic NR2B-containing NMDA receptors. Excitotoxic neuronal death was induced in cortical neurons by bath application of NMDA (50 μM, 20 min) after the blockade of synaptic NMDA receptors with MK-801 plus bicuculline, and cell death was assayed 20 h later. NMDA elicited neuronal apoptosis, which was exacerbated when the NR2B-containing component was selectively stimulated (NVP+NMDA), but eradicated when the NR2A-containing component was specifically activated (Ro+NMDA). ** p<0.001 compared with control. # p<0.05, ## p<0.001 compared with NMDA treatment. n=11-12 from two separate experiments for each group.

FIG. 5. Selective activation of NR2A-containing NMDA receptors protects neurons from NMDA receptor- or non-NMDA receptor-mediated neuronal apoptosis. A. Activation of extrasynaptic NR2A-containing NMDA receptors can counteract the NMDA receptor-independent apoptosis. Bath application of staurosporine (STS, 100 nM, 1 h), after blockade of synaptic NMDA receptors with pre-treatment of MK-801 plus bicuculline and of extrasynaptic NR2B receptors in the presence of Ro 25-6981 (Control), induced a significant increase in neuronal apoptosis (STS). Brief application of NMDA (200 μM, 5 min) did not produce neuronal apoptosis on its own (NMDA), but significantly reduced the STS-induced neuronal apoptosis (NMDA+STS) and the NMDA-induced neuroprotective action was abrogated by co-application of NVP-AAM077 (0.4 μM; NVP+NMDA+STS). ** p<0.001 compared with Ro 25-6981 treatment. ## p<0.001 compared with STS treatment. n=8-12 for each group from three separate experiments. B. Pretreatment of neuronal cultures with glycine (300 μM plus strychnine 10 μM) for 10 min significantly reduced neuronal apoptosis produced by NMDA applied thereafter (Gly+NMDA). This neuprotective effect was abolished by co-application of NR2A antagonist NVP-AAM077 (0.4 μM) with glycine (NVP+Gly+NMDA), but not by co-application of NR2B-specific antagonist Ro25-6981 (0.5 μM) with glycine (Ro+Gly+NMDA), indicating that the neuroprotective effect of glycine is primarily mediated through enhancing the activation of NR2A-containing NMDARs. * p<0.05, *** p<0.001 compared with control. # p<0.05, ## p<0.01 compared with NMDA. n=17-18 for each group from three separate experiments.

FIG. 6. Pretreatments with NR2A- and NR2B-specific antagonists respectively promote neuronal survival and death in both in vitro and in vivo models of ischemia. A. NR2A- and NR2B-containing receptors exert opposing effects in ischemic neuronal injuries in vitro. Cortical cultures were challenged with a 1-h oxygen and glucose deprivation (OGD) and apoptosis was assayed 23 h after the challenge. OGD resulted in a significant increase in neuronal apoptosis compared with non-challenged controls (Control) and the OGD-induced apoptosis was respectively potentiated by the NR2A specific antagonist NVP-AAM077 (NVP+OGD; 0.4 μM) and inhibited by the NR2B antagonist Ro 25-6981 (Ro+OGD; 0.5 μM) when bath applied 30 min prior to, and during, the OGD challenge. ** p<0.001 compared with control. # p<0.05, ## p<0.001 compared with OGD. n=17-18 for each group from three separate experiments. B and C. NR2A- and NR2B-containing receptors exert opposing effects in ischemic neuronal injuries in vivo. Adult rats were subjected to a 1-h focal cerebral ischemia produced by middle cerebral artery occlusion (MCAo), and cerebral infarction was assessed 24 h after MCAo onset. Intravenous infusion 30 min before MCAo onset of NVP-AAM077 (NVP+MCAo; 2.4 mg/kg; n=5) and Ro 25-6981 (Ro+MCAo; 6 mg/kg; n=6) respectively increased and decreased both infarct area (B) and total infarct volume

(C). * p<0.05, ** p<0.001 compared with MCAo. D. Neurological scores assessed 24 h after stroke onset in the same groups of animals shown in (B) and (C) indicate that blockade of the NR2A-containing NMDA receptors resulted in a trend toward worsening neurological function, whereas blockade of NMDA receptors containing NR2B markedly improved neurological behavior. ** p<0.001 compared with MCAo.

FIG. 7 Post-ischemic potentiation of NR2A-containing NMDA receptors through administration of glycine reduces ischemic brain damage in an in vivo focal ischemic stroke model. Adult rats received either drug or saline treatment 3 h after a 1.5-h MCAo challenge (4.5 h after MCAo onset). A. General blockade of NMDA receptors with non-subunit specific antagonist MK801 (MK801; 1 mg/Kg; n=8) following stroke was no longer neuroprotective, whereas a post-stroke treatment with NMDA receptor co-agonist glycine (Gly; 800 mg/kg; n=8) significantly reduced total infarct volume. The glycine effect was fully blocked by co-application of MK-801 (Gly+MK-801; n=7), indicating the mediation by NMDA receptors. * p<0.05. compared with Control (MCAo alone) B. Further experiments showed that similar to MK801, post-ischemic treatment with NR2B selective antagonist Ro 25-6981 (Ro; 6 mg/kg; n=10) was ineffective while the glycine effect persisted in the presence of Ro 25-6981 (6 mg/kg) (Gly+Ro; n=9). The addition of NR2A antagonist NVP-AAM066 (2.4 mg/kg) (Gly+Ro+NVP; n=10) abolished the neuroprotection offered by glycine, indicating glycine acts through selective potentiation of NR2A-containing NMDA receptors. *** p<0.001 compared with Control (MCAo alone) C. Representative rat brain sections stained with hematoxylin and eosin (H & E) from each treatment group in B. Pale staining indicates infarct.

### DETAILED DESCRIPTION

As set out in more detail in the following Examples, in mature cortical cultures, activation of either synaptic or extrasynaptic NR2B-containing NMDA receptors results in excitotoxicity, increasing neuronal apoptosis. In contrast, in accordance with various aspects of the invention, activation of either synaptic or extrasynaptic NR2A-containing NMDA receptors, relative to NR2B-containing receptors, promotes neuronal survival and exerts a neuroprotective action against both NMDA receptor- and non-NMDA receptor-mediated neuronal damage.

Evidence from an in vivo rat model of focal ischemic stroke showed that an NR2A antagonist increased infarct volume, while administration of an NR2A-containing NMDA receptor agonist, glycine, to selectively activate NR2A-containing NMDA receptors, attenuated ischemic brain damage (even when delivered 4.5 h following stroke onset). Accordingly, in various aspects, the invention provides for neuroprotective enhancement of NR2A-containing NMDA receptor activation.

In keeping with various aspects of the invention, it has been demonstrated that NR2A- and NR2B-containing NMDA receptors exert differential roles in mediating NMDA-induced neuronal death. This was demonstrated in rat cortical cultures of 11-14 days in vitro (DIV) using subunit-specific NMDA receptor antagonists, NVP-AAM077 which preferentially inhibits NR2A-containing receptors at the concentration of 0.4-1 μM (Liu et al., 2004; Massey et al., 2004; Tigaret et al., 2006) and Ro25-6981, which specifically blocks NR2B-containing receptors (Mutel et al., 1998; Fischer et al., 1997).

It is also demonstrated herein that both subtypes of NMDA receptors exist in these neurons, and NVP-AAM077 and Ro25-6981 function as respective subunit-selective antagonists. To illustrate this, we examined the ability of these antagonists to inhibit whole-cell currents evoked with a rapid and brief application of NMDA (50 μM NMDA, 10 μM glycine, 5 μM strychnine).

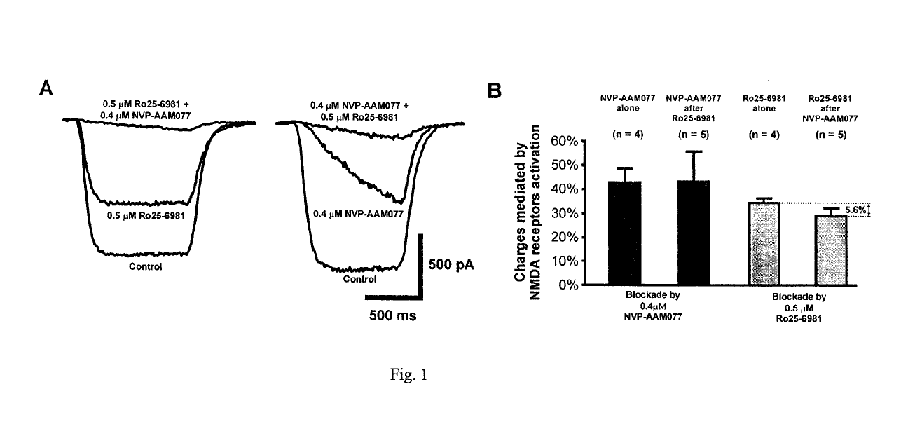

As shown in FIG. 1A, bath application of either NVP-AAM077 (0.4 μM) or Ro25-6981 (0.5 μM) alone produced a partial, but significant, blockade of the NMDA-induced currents. The two antagonists were also applied sequentially, to compare the degree of blockade produced by each antagonist when it was applied alone and applied following the blockade by the other antagonist (FIGS. 1A and B). NVP-AAM077 produced similar blockade when applied either alone (42.9%±5.9%) or after Ro25-6981 blockade (43.4%±12.4%), confirming that Ro25-6981 at the concentration used herein is a very specific to NR2B subunit antagonist, with little effect toward to the blockade of NR2A-containing receptors (Mutel et al., 1998; Fischer et al., 1997) and that NVP-AAM077 effectively blocking NR2A-containing receptor-mediated current (Liu et al., 2004; Massey et al., 2004; Tigaret et al., 2006). Following NVP-AAM077 blockade, the percentage of NMDA current inhibition by Ro25-6981 was reduced by a proximately 5.6% (34.6%±1.8% when applied alone vs 29.0%±3.3% after NVP, P>0.05). The reduction may reflect a small degree of cross-inhibition of NR2B receptors by NVP-AAM077 in these neurons under our experimental conditions. However, several recent studies have demonstrated that such a small percentage of contaminant NR2B inhibition may not significantly affect the utility of NVP-AAM077 as a NR2A-subunit preferential antagonist (Liu et al., 2004; Massey et al., 2004; Tigaret et al., 2006). Together, our results indicate that both NR2A- and NR2B-containing receptor subtypes are expressed in these neurons and the two antagonists selectively block respective receptor subtypes with little cross-receptor subtype antagonism.

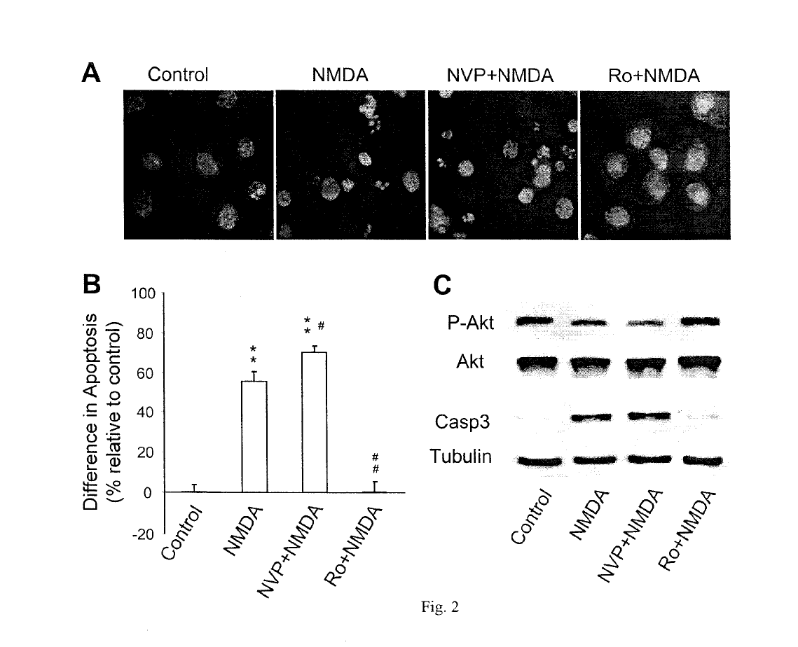

Having established the co-existence of both subtypes of NMDA receptors and the specificity of the antagonists to respectively inhibit these receptor subtypes, we examined the effects of these subunit-specific NMDA receptor antagonists on NMDA receptor-mediated neuronal death. NMDA-mediated neuronal death was induced by incubating neuronal cultures with 50 μM NMDA plus 10 μM glycine for 20 min (NMDA-mediated excitotoxicity). Neuronal injuries were determined 20 h after treatment by nucleus staining with Hoechst-33342. NMDA treatment induced neuronal injuries as indicated by an increase in the proportion of neurons displaying nuclear condensation and/or fragmentation (FIG. 2A). Neuronal apoptosis was confirmed using a quantitative biochemical measurement of intranucleosomal fragmentation (FIG. 2B). The NMDA-induced neuronal damage was a result of the specific activation of NMDA receptors, as it was fully blocked by the NMDA receptor antagonist, APV (50 μM; data not shown).

To illustrate the individual roles of NR2A- and NR2B-containing NMDA receptor subtypes in NMDA-induced neuronal apoptosis, we compared the effects of a blockade of these receptors with subunit-specific antagonists. Bath application of NR2B antagonist Ro25-6981 (0.5 μM) prevented NMDA-induced neuronal apoptosis, indicating the critical involvement of this NMDA receptor subtype. In striking contrast, we found that application of NR2A subunit-specific antagonist NVP-AAM077 (0.4 μM) failed to block, and in fact significantly enhanced, NMDA-induced apoptosis (FIGS. 2A, B; p<0.05 compared with NMDA alone). These unexpected results indicate that activation of NR2A-containing NMDA receptors exerts a cell survival promoting effect that counteracts the apoptotic action produced by NR2B-containing receptors.

The opposing actions of NR2A and NR2B were further confirmed by characterization of biochemical signals involved in mediating cell survival and apoptotic death. The serine/threonine kinase Akt/PKB is a cell-survival promoting molecule (Dudek et al., 1997) and inhibition of this kinase activity contributes to NMDA receptor-mediated apoptosis (Chalecka-Franaszek et al., 1999). As shown in FIG. 2C, treatment of neurons with NMDA resulted in a significant reduction in Akt kinase activity as gauged by the dephosphorylation of S473 (Wang et al., 2004; Coffer et al., 1998). Blocking NR2A receptors with NVP not only failed to prevent, but slightly increased the NMDA-induced reduction in Akt activity. In contrast, following NR2B blockade, the NMDA-induced Akt inhibition was virtually eliminated (FIG. 2C). Activation of certain caspases, such as caspase-3 and -7 (Wang et al., 2004; Okamoto et al., 2002), has been suggested to be a critical step in NMDA-induced neuronal apoptosis. In the present study, we found that NMDA treatment dramatically increased the level of the activated form of caspase-3, as shown by western blots using an antibody that specifically recognizes activated/cleaved caspase-3 (FIG. 2C). The activation of the caspase-mediated death signal was inhibited by blocking NR2B receptors, but slightly enhanced following NR2A blockade. Thus, activation of NR2A- or NR2B-containing NMDA receptors have opposing impacts on cell survival and apoptotic signal pathways, thereby differentially promoting neuronal survival and death.

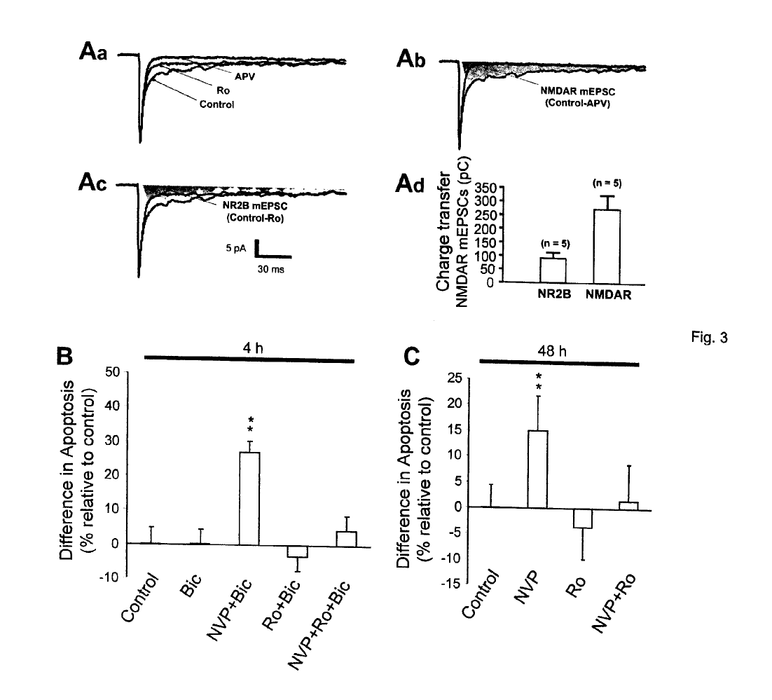

To differentiate the effects of the NMDA receptor subunit compositions from their anatomical localizations, we functionally mapped the expression of NR2A- and NR2B-containing NMDA receptors at synaptic and extrasynaptic sites, to illustrate their roles in promoting cell survival or death in cultured cortical neurons following pharmacological isolation. Although the vast majority of synaptic NMDA receptors are NR2A-containing, we have demonstrated that functional NR2B-containing receptors are also expressed at the synaptic sites of the cultured cortical neurons used in the present Examples, using whole-cell recording of spontaneous miniature excitatory postsynaptic currents (mEPSCs). As shown in FIG. 3A, under these recording conditions mEPSCs are comprised of both a fast, α-amino-3-hydroxy-5-methyl-4-isoxazole propionic acid (AMPA) subtype glutamate receptor-mediated component, which was completely blocked by the non-NMDA receptor antagonist DNQX (data not shown), and a slow, NMDA receptor-mediated component which was fully blocked by the NMDA receptor antagonist APV (FIGS. 3Aa, Ab, Ad). Consistent with the presence of a proportion of functional synaptic NR2B-containing receptors, the NMDA component was significantly reduced by bath application of NR2B antagonist Ro 25-6981 (0.5 μM; FIGS. 3Ac, Ad). As mEPSCs are primarily mediated by synaptically localized receptors activated by glutamate spontaneously released from presynaptic terminals, the sensitivity to NR2B antagonist demonstrates that functional NR2B-containing NMDA receptors are present within the glutamatergic synapses of the neurons used in the present Examples. On average, the NR2B-containing receptor-mediated component accounted for 32.4±3.6% of the synaptic NMDA currents (n=5; FIGS. 3Ac, Ad) and the remainder was primarily mediated by NR2A-containing receptors as it was largely eliminated in the presence of the NR2A-specific antagonist NVP-AAM077 (0.4 μM; n=5). Thus, similar to hippocampal CA1 neurons in brain slices (Liu et al., 2004; Wong et al., 2005), functional subpopulations of both NR2A- and NR2B-containing NMDA receptors, although the former is predominant, are expressed at the synapses of the cultured neurons used in the present Examples.

The function of the NR2A and NR2B synaptic receptor subpopulations in mediating neuronal survival or death is also illustrated herein, as follows. To increase activation of synaptic NMDA receptors by synaptically released glutamate, neurons were incubated with the GABAA receptor antagonist bicuculline (50 μM, 4 h). Bicuculline increases neuronal excitation by blocking the GABAA receptor-mediated synaptic inhibition and thereby enhances action potential-dependent synchronized release of glutamate from presynaptic terminals. Neuronal apoptosis was quantified 20 h following the treatments. We demonstrate that stimulation of synaptic NMDA receptors by application of bicuculline alone, or in the presence of NR2B antagonist Ro 25-6981, did not cause apoptotic cell death (FIG. 3B). In contrast, blocking synaptic NR2A-containing receptors by co-application of NVP-AAM077 with bicuculline significantly increased neuronal apoptosis (p<0.001; FIG. 3B). The NR2A blockade-induced neuronal apoptosis was demonstrably mediated by synaptic NR2B-containing receptors, as it was prevented in the presence of Ro 25-6981 (p<0.01; FIG. 3B).

Under bicuculline incubation, the increased action potential-dependent synaptic release of glutamate may lead to activation of extrasynaptic NMDA receptors by glutamate spillover. Accordingly, we also illustrate the impact of a blockade of synaptic NMDA receptor activation by glutamate spontaneously released from terminals under basal, non-stimulated conditions. Incubation of neurons with NVP-AAM077 for 4 h failed to increase neuronal apoptosis (data not shown). However, when the incubation time was increased to 48 h, a significant increase in neuronal apoptosis was observed (FIG. 3C, p<0.01). The synaptic NR2A antagonist-induced apoptosis was also prevented by the blockade of synaptic NR2B receptors with Ro 25-6981. In contrast, blockade of synaptic NR2B alone for up to 48 h did not increase neuronal apoptosis (FIG. 3C). Together, these results illustrate that both synaptic NR2A- and NR2B-containing receptors are activated by spontaneously released glutamate from the presynaptic terminal and hence tonically exert opposing influences with respect to promoting cell survival or death. Under typical physiological conditions, the NR2A-mediated cell survival-promoting effect counteracts the tonic apoptotic action of NR2B, thereby maintaining normal neuronal survival. Synaptic NR2B-mediated neuronal death is unmasked after pharmacological blockade of the NR2A-mediated cell survival signaling pathway.

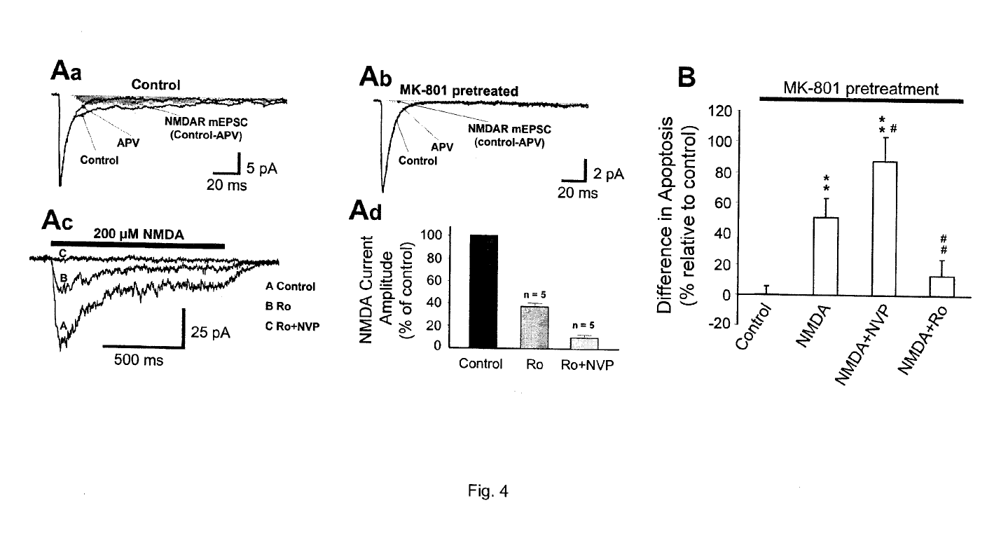

In contrast to the predominant expression of NR2A-containing receptors at synapses, NR2B-containing receptors are thought to be the predominant NMDA receptor expressed at extrasynaptic sites in mature neurons (Massy et al., 2004; Tovar et al., 2002). To determine if some, albeit small, proportion of extrasynaptic NMDA receptors contain NR2A in the neurons under study, we first pharmacologically blocked all NMDA receptors expressed at synapses and then examined whether currents gated through extrasynaptic NMDA receptors are sensitive to NR2A subunit-specific antagonism. The selective blockade of synaptic NMDA receptors was achieved by co-application of bicuculline (50 μM) and MK-801 (10 μM) for 10 min. Bicuculline enhances synaptic release of glutamate and thereby selectively activates synaptic NMDA receptors (Hardingham et al., 2002). MK-801, as an irreversible blocker of open NMDA receptor channels (Tovar et al., 2002; Huettner et al., 1988), can only block the bicuculline-activated synaptic NMDA receptors, and cannot block extrasynaptic channels that are not activated during bicuculline application. The complete blockade of synaptic NMDA receptors could be achieved within 10 min of bicuculline and MK-801 co-application as indicated by the virtual elimination of the slow, APV-sensitive component of mEPSCs (FIGS. 4Aa, Ab). Little recovery was observed one hour following wash-out of the drugs. The currents gated through extrasynaptic NMDA receptors were then induced by application of NMDA (200 μM) via a fast perfusion system after washing out bicuculline and MK-801. The extrasynaptic NMDA receptor-mediated currents were largely reduced by the NR2B antagonist Ro 25-6981 (FIGS. 4Ac, Ad), consistent with the finding that extrasynaptic NMDA receptors are predominantly NR2B-containing. The residual, NR2B antagonist-resistant current was virtually completely blocked by the NR2A antagonist NVP-AAM077 (FIGS. 4Ac, Ad), indicating that the non-NR2B-containing extrasynaptic NMDA receptors were largely NR2A-containing receptors. On average, about 26.6±2.3% (n=5) of total currents gated by extrasynaptic NMDA receptors were mediated by NR2A-containing receptors (FIG. 4Ad). These results illustrate the existence of a substantial number of functional extrasynaptic NR2A-containing NMDA receptors in mature cultured cortical neurons.

In accordance with various aspect of the invention, we have illustrated the role of extrasynaptic NR2A- and NR2B-containing receptors in mediating NMDA-induced cell survival and death. After a specific blockade of synaptic NMDA receptors and wash-out of bicuculline and MK-801, the neurons were treated with NMDA (50 μM plus 10 μM glycine) for 20 min in the absence or presence of NVP-AAM077 (0.4 μM) or Ro 25-6981 (0.5 μM). Quantitative neuronal apoptosis assays performed 20 h after the treatments showed that NMDA application alone (non-selective activation of extrasynaptic NMDA receptors) elicited significant apoptosis (p<0.001, FIG. 4B) which could be prevented by a selective blockade of NR2B-containing extrasynaptic NMDA receptors with Ro 25-6981. In sharp contrast, blockade of the NR2A-containing receptors with NVP-AAM077, i.e. leaving NR2B-containing NMDA receptors intact, did not prevent, but instead potentiated NMDA-mediated apoptosis (p<0.05 compared with NMDA treatment). Thus, as with synaptic NMDA receptors, activation of extrasynaptic NR2A-containing receptors has a role in promoting cell survival, counteracting NR2B-containing receptor-mediated neuronal apoptosis. Taken together, the data illustrated in FIGS. 3 and 4 illustrate that, regardless of their anatomical locations (synaptic vs. extrasynaptic), NR2A- and NR2B-containing receptors are capable of having opposing roles in mediating NMDA-elicited neuronal survival and apoptosis.

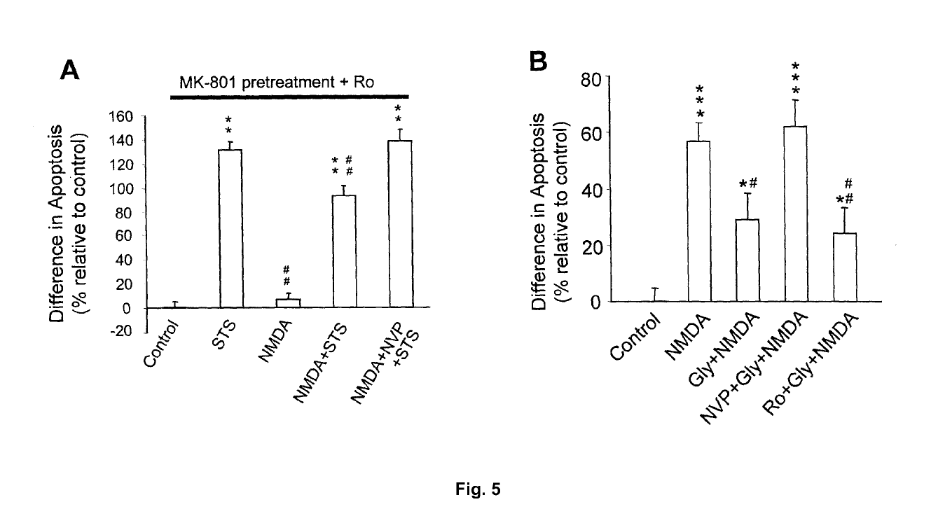

In alternative aspects of the invention, we have achieved specific activation of NR2A-containing receptors using two different strategies. First, we examined the effect of selective activation of extrasynaptic NR2A-containing receptor activation on neuronal appopotosis induced by staurosporine (STS), a potent apoptosis inducer (Budd et al., 2000). In these embodiments, all synaptic NMDA receptors were irreversibly blocked by pretreatment of the neurons with co-application of bicuculline and MK-801, and extrasynaptic NR2B-containing NMDA receptors were blocked by addition of Ro 25-6981 (0.5 μM) in the medium through out the experiments. As shown in FIG. 5A, STS (100 nM, 1 h) treatment triggered tremendous neuronal apoptosis. Bath application of NMDA (200 μM, 5 min) did not increase neuronal apoptosis on its own, confirming the effective blockade of NR2B-containing receptor-mediated apoptotic actions by Ro 25-6981. However, the application of NMDA, which would primarily activate extrasynaptic NR2A-containing receptors under these conditions, significantly reduced STS-induced apoptosis (p<0.001 compared with STS alone; FIG. 5A). The NMDA-induced neuronal protection was indeed mediated by NR2A-containing receptors as it was prevented by co-application of NVP-AAM077 (p<0.001 compared with STS alone).

In an alternative approach, we illustrate the effect of enhancement of synaptic NR2A activation on reducing NMDA-induced excitotoxicity. We accomplished the selective enhancement of synaptic NMDA receptor activation by a brief bath application of supra-saturating concentration of glycine (Lu et al., 2001; Man et al., 2003). As an NMDA receptor co-agonist (McBain et al., 1994), glycine applied through bath can enhance the function of synaptic NMDA receptors that are activated by glutamate spontaneously released from presynaptic terminal under non-stimulated conditions, but not of extrasynaptic NMDA receptors which are not activated under the non-stimulated condition (Lu et al., 2001; Man et al., 2003). Taking advantage of the fact that synaptic NMDA receptors in these neurons are predominantly NR2A-containing and their activation produces a dominant cell survival promoting action (FIG. 3), we have illustrated that selective enhancement of activation of synaptic NMDA receptors with supra-saturating concentration of glycine leads to an increase in synaptic NR2A-dependent neuronal survival. In this embodiment, a 10 min pretreatment of neurons with glycine (300 μM; plus 10 μM strychnine to block glycine CI-channel) significantly reduced NMDA-induced neuronal apoptosis (p<0.05, compare with NMDA treatment alone; FIG. 5B). The survival-promoting effect of glycine was indeed mediated by activation of the synaptic NR2A-containing NMDA receptor subpopulation, as the neuroprotective effects of glycine pretreatment was prevented by NR2A antagonist NVP-AAM077, but not by affected by NR2B antagonist Ro25-6981 (FIG. 5B). Together these results illustrate that preferential activation of NR2A receptors compared to NR2B receptors induces a pro-survival pathway that is able to guard against both NMDA receptor- and non-NMDA receptor (such as STS)-mediated neuronal damage.

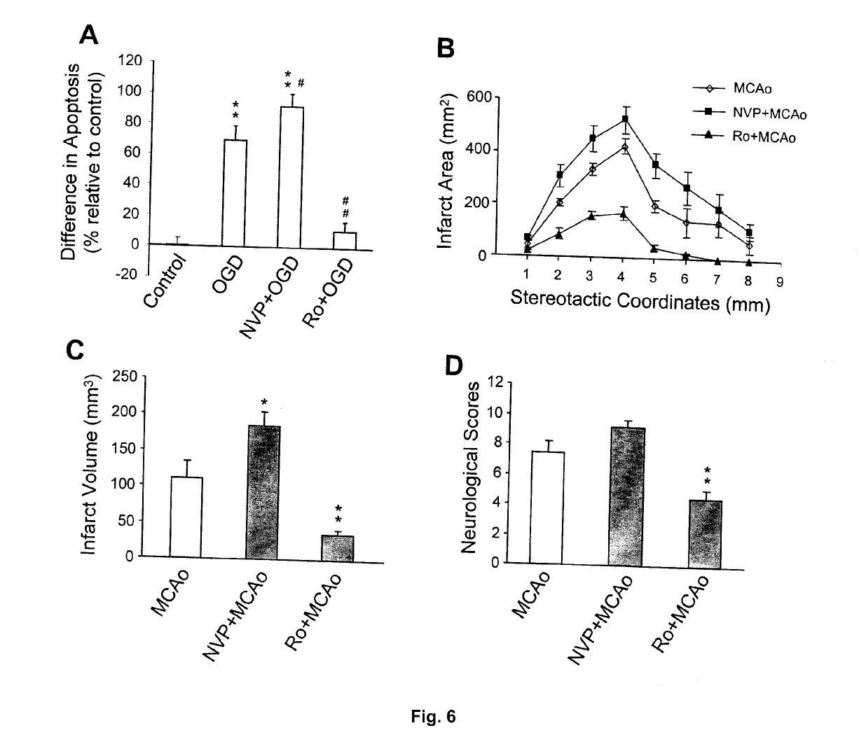

Taking advantage of the finding of the opposing roles of NR2A- and NR2B-containing NMDA receptors in mediating cell survival and death, one aspect of the invention involves modulating the activity of the two subpopulations of receptors to ameliorate neuronal injury following acute brain insults, such as stroke and brain trauma. To illustrate this aspect of the invention, we employed a well-characterized in vitro stroke model, oxygen and glucose deprivation (OGD) (Goldberg et al., 1993; Aarts et al., 2002). Cortical cultures of 11-14 DIV were exposed to an anaerobic atmosphere for 1 h in a glucose-free solution in the absence or the presence of either NVP-AAM077 (0.4 μM) or Ro 25-6981 (0.5 μM). Neuronal apoptosis was quantitatively determined 20 h after OGD. As shown in FIG. 6A, 1 h of OGD was able to produce a pronounced increase in neuronal apoptosis. Selective inhibition of the NR2A-containing NMDA receptors with NVP-AAM077 significantly enhanced OGD-induced neuronal apoptosis (p<0.05 compared with OGD), and in contrast, a specific blockade of the NR2B-containing NMDA receptors by Ro 25-6981 drastically reduced the ODG-induced apoptosis (p<0.001 compared with OGD; FIG. 6A).

This aspect of the invention was also illustrated in vivo using a rat focal ischemic stroke model—middle cerebral artery occlusion (MCAo) (Aarts et al., 2002; Bederson et al., 1986). We first infused NVP-AAM077 (2.4 mg/kg), Ro 25-6981 (6 mg/kg (Loschmann et. al., 2004)) or vehicle (saline) intravenously in the rats 30 min prior to stroke onset. The animals were then subjected to a 1-h transient ischemic stroke induced by MCAo. This relatively short duration of ischemia was chosen to unmask the potential neuroprotective effects mediated by NR2A-containing receptors activated during the stroke challenge. Neurological score and cerebral infarction were examined 24 h after the MCAo onset. Similar to the results observed with OGD in vitro, we found that blockade of NR2A-containing NMDA receptors significantly increased the infarct areas and the total infarct volume, whereas, in sharp contrast, the stroke-induced brain injuries were remarkably reduced by NR2B antagonism (FIGS. 6B, C). Specifically, when compared with saline-treated animals, NVP-AAM077 pre-treatment gave rise to a 67.0±17.9% increase in total infarct volume (n=5; p<0.05), while Ro 25-6981 treatment decreased the total infarct volume by 67.8±4.3% (n=6; p<0.01). Neurological behavioral tests showed that the NVP-AAM077-treated animals exhibited a trend toward poorer neurological function while Ro 25-6981 treatment produced a significant protective effect (FIG. 6D). Together, these observations indicate that both NR2A- and NR2B-containing NMDA receptor subtypes are activated during stroke, exerting opposing effects on ischemic brain damage.

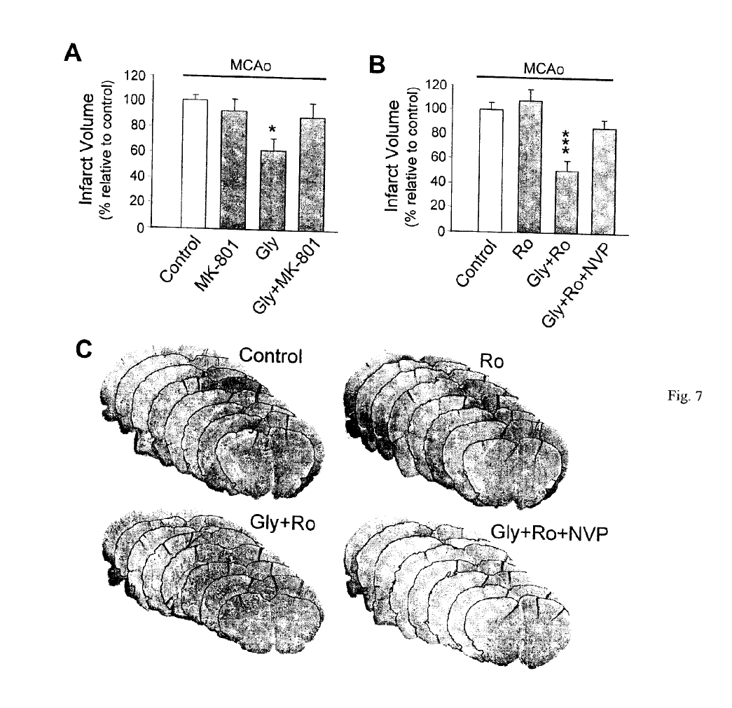

In some clinical settings, it is desirable to implement therapy after the onset of neuronal injury, such as stroke or other ischemic events. In accordance with one aspect of the invention, we therefore illustrate the effects of post-ischemic blockade of NR2B or potentiation of NR2A in reducing ischemic brain injury. In this aspect of the invention, preferential, relative or selective activation of NR2A-containing receptors may be used to initiate cell survival promoting signals, protecting neurons against ischemic damage following the pathology-inducing event. The data herein show that treatment with non-subunit specific NMDA receptor antagonist MK801 (1 mg/Kg (Margaill et al., 1996); FIG. 7A) 3 h following a 1.5-h MCAo challenge (4.5 h after stroke onset) did not provide any noticeable neuroprotection when compared MCAo alone (FIG. 7A). In contrast, selective activation of NR2A-containing receptors with the application of NMDA receptor co-agonist glycine (800 mg/Kg) resulted in a remarkable reduction in total infarct volume (FIG. 7A). The glycine effect is mediated through enhancement of synaptic NMDA receptors and not its action on glycine receptor-mediated CI-channels, as it was virtually completely abolished by co-administration of MK801 (FIG. 7A). The narrow therapeutic time window of NMDA receptors antagonists in the treatment of stroke was also confirmed by the ineffectiveness of NR2B specific antagonist Ro 25-6981 (6mg/Kg; FIG. 7B). Moreover, the glycine effect was resistant with NR2B antagonist Ro 25-6981, but prevented by NR2A antagonist NVP-AAM077, demonstrating the efficacy of glycine in mediating the specific enhancement of NR2A-containing NMDA receptor-mediated cell survival (FIGS. 7B and C). These results indicate that post-ischemic potentiation of the pro-survival action of NR2A-containing NMDA receptors is an effective neuroprotection therapy.

The results illustrated herein demonstrate that NMDA receptor activation can produce either neuronal survival or death promoting action, and that this dual action is dictated by receptor subunit composition and not subcellular localization (synaptic vs. extrasynaptic). The cell survival action can be blocked by the NR2A preferential antagonist NVP-AAM077. The lack of blockade of the NMDA receptor-mediated cell survival action by the NR2B antagonist Ro 25-6981 essentially rules out the contribution of this subunit. On the other hand, the efficient blockade of NMDA receptor-dependent cell death by Ro 25-6981, but not by NVP-AAM077, strongly suggest that it is the NR2B-containing, but not NR2A-containing, NMDA receptor subpopulation that plays a primary role in triggering intracellular cascades that leading to NMDA- or ischemia-induced neuronal apoptosis. The lack of effect in blocking the cell death by NVP-AAM077 also further indicates that a small fraction of NR2B inhibition provided by this antagonist is not sufficient to block the NR2B-dependent cell death.

In accordance with various aspects of the invention, the net impact of NMDA receptor activation on neuronal survival and death is dictated by modulating the balance between the activation of NR2A- and NR2B-containing NMDA receptor subpopulations. In alternative embodiments, the precise nature of the required receptor subtype modulation may vary, for example depending on developmental stage of the subject, brain areas or conditions to be treated. As demonstrated in the present work, NR2A-containing receptor activation, in addition to counteracting NR2B-containing receptor-mediated cell death, has the ability to guard against non-NMDA receptor-mediated apoptotic processes.

In one aspect of the invention, we demonstrate that the NMDA receptor-mediated excitotoxic neuronal injuries following stroke in the rat MCAo model of focal ischemia are primarily mediated by NR2B-containing receptors, as NR2B-containing NMDA receptor-specific antagonist applied prior to the stroke onset significantly reduced the brain damage. However, the NR2B antagonist, on its own, appears to have a relatively narrow therapeutic window since it offers little protection when administered 4.5 h after the stroke onset. Thus, NR2B specific antagonists, on their own, would be expected to have no effect after this point. Administration of non-subunit specific NMDA receptor antagonists such as MK-801 and amantadine at this point may even be harmful due to their blockade of NR2A-containing receptor-dependent pro-survival signaling. Unfortunately, in most clinical settings, due to the time required to transport a patient to the hospital and obtain a definitive diagnosis, treatment is not usually possible until several hours after the onset of neuronal injury, which may be outside the window of efficacy for NMDA receptor blockers, on their own, but within the window for treatments in accordance with various aspects of the invention.

Activation of NR2A-containing NMDA receptors in accordance with alternative embodiments of the invention may be implemented so as to achieve particular advantages over previously proposed NMDA receptor antagonism-based therapies. For example, as demonstrated herein, therapies in accordance with the invention may have a broader therapeutic window than NR2B-containing receptor blockade therapies alone. In addition, NR2A-containing receptor activation therapies of the invention may be effective not only against NMDA receptor-mediated cell death (primary neuronal injuries), but also in treatment of non-NMDA receptor-mediated cell death (secondary neuronal injuries). In addition to the neuronal injuries caused by acute brain insults such as stroke and brain trauma, utilization of NR2A-containing receptor-dependent pro-survival signaling may also be an effective neuroprotective therapy for a number of chronic neurodegenerative disorders, such Parkinson's disease, Huntington's disease, amyotrophic lateral sclerosis and Alzheimer's disease, where a “slow” NMDA receptor-mediated excitotoxicity has been implicated (Lipton et al., 1994; Ikonomidou et al., 2002; Zoghbi et al., 2000).

In various aspects of the invention, a relative enhancement of NR2A-containing NMDA receptor function, compared to NR2B function, may be achieved by the combination of a non-subunit specific NMDA receptor enhancer, such as glycine, and an NR2B specific antagonist. NMDA receptor glycine site agonists, such as D-cycloserine (Posey et al., 2004), and NR2B specific antagonists (Chazot, 2000) are generally available. Examples of NMDA receptor glycine site agonists include D-cycloserine (Posey et al., 2004). Examples of NR2B specific antagonists include ifenprodil and Ro 25-6981. (Chazot, 2000). Useful chemical entities may include agonists of NR2A-containing NMDA receptors or antagonists of NR2B-containing NMDA receptors, and include those that modulate the expression, activity or stability of the NR2A- or NR2B-containing NMDA receptor. To identify such compounds, NR2A or NR2B expression, biological activity, or an effect of such expression or activity such as cell survival or signal transduction is measured following the addition of candidate compounds to a culture medium of neuronal cells expressing NR2A- and/or NR2B-containing NMDA receptors. Alternatively, the candidate chemical entities may be directly administered to an animal model such as a rat MCAo stroke model, and candidate chemical entities may be identified by their effect on neuronal survival or death. An NR2B-containing NMDA receptor antagonist administered in combination with glycine, or an NR2A-containing NMDA receptor agonist are two examples of solutions to this current unmet need for temporally flexible brain trauma and stroke therapeutics. An added advantage to the application of an NR2A-containing NMDA receptor agonist as a therapeutic may be the subsequent selective activation of cell survival pathways

In alternative aspects, the invention provides methods for identifying chemical entities for use in various aspects of the invention, such as selective agonists of NR2A-containing NMDA receptors. In some embodiments, recombinant a NR1/NR2A heteromeric complexes may for example be utilized (Chu et al., 1995; Yamada et al., 2002). In some embodiments, NMDA receptors (such as NR1/NR2A containing receptors) may be expressed in vitro, either in well-established cell lines (e.g., HEK 293) or in primary Xenopus oocytes (Stern et al., 1992; Priestley et al., 1995; Bresink et al., 1996; Grimwood et al., 1996). In some aspects of these screening methods, NMDA agonist activity can be measured using whole-cell voltage-clamp electrophysiology (Mayer et al., 1987; Priestley et al., 1995; Losi et al., 2006). In accordance with the foregoing techniques, one or more cell lines (or Xenopus oocytes) that expresses NR1/NR2A may be used in combination with whole-cell voltage electrophysiology readings to screen for selective agonists of NR2A-containing receptors. In some embodiments, controls for screening methods may be provided, including NR1/NR2B transfected cells or cell lines, for use in comparisons of activity (Yang et al., 2001).

In addition to direct NMDA receptor modulation by ligand binding, there are a variety of alternative approaches to modulating NMDA receptor activity in accordance with alternative embodiments of the invention, such as the modulation of downstream signaling. For example: inhibition of direct binding between NR2B and CaMKII at the S-site and T-site; inhibition of phosphorylation of NR2B by CaMKII (Bayer et al., 2006; increasing the levels of phospho-CREB (Ser-133) (Amadoro et al., 2006); blocking the association between NR2B and SynGap (Kim et al., 2005); blocking re-uptake of an NR2A-containing receptor agonist, such as glycine or D-serine (Kemp et al., 2002).

Formulations and Medicaments

A medicament is a chemical entity capable of producing an effect, that may be administered to a patient or test subject. The effect may be chemical, biological or physical, and the patient or test subject may be human, or a nonhuman animal, such as a rodent or transgenic mouse. The medicament may be comprised of the effective chemical entity alone or in combination with a pharmaceutically acceptable excipient.

The medicaments of the present invention may be formulated for administration by any of various routes. The medicaments may include an excipient in combination with the effective chemical entity, and may be in the form of, for example, tablets, capsules, powders, granules, lozenges, pill, suppositories, liquid or gel preparations. Medicaments may be formulated for parenteral administration in a sterile medium. The medicament may be dissolved or suspended in the medium. Medicaments may be formulated for a subdermal implant in the form of a pellet, rod or granule. The implant or implants may be inserted subcutanerously by open surgery or by use of a trochar and cannula under local anaesthesia. The implant may be periodically replaced or removed altogether. Medicaments may also be formulated for transdermal administration using a patch. The patch is applied to a shaven area of the skin of the patient while the medicament is desired for administration, and removed when no longer needed. Conventional pharmaceutical practice may be employed to provide suitable formulations or compositions to administer the compounds to patients suffering from or presymptomatic for neurological damage or neural dysfunction. Compounds may be administered systemically or may be administered directly to the CNS or other region of neurological damage. In some embodiments, compounds according to the invention may be provided in a form suitable for delivery across the blood brain barrier. Any appropriate route of administration may be employed, for example, parenteral, intravenous, subcutaneous, intramuscular, intracranial, intraorbital, ophthalmic, intraventricular, intracapsular, intraspinal, intracisternal, intraperitoneal, intranasal, aerosol, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found in, for example, “Remington's Pharmaceutical Sciences” (19th edition), ed. A. Gennaro, 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. A pharmaceutically acceptable excipient includes any and all solvents, dispersion media, coatings, antibacterial, antimicrobial or antifungal agents, isotonic and absorption delaying agents, and the like that are physiologically compatable. The excipient may be suitable for intravenous, intraperitoneal, intramuscular, intrathecal or oral administration. The excipient may include sterile aqueous solutions or dispersions for extemporaneous preparation of sterile injectable solutions or dispersion. Use of such media for preparation of medicaments is known in the art.

For therapeutic or prophylactic compositions, the compounds are administered to an individual in an amount sufficient to stop or slow cell neuronal degeneration or apoptosis. An “effective amount” of a compound according to the invention includes a therapeutically effective amount or a prophylactically effective amount. A “therapeutically effective amount” refers to an amount effective, at dosages and for periods of time necessary, to achieve the desired therapeutic result, such as reduction of neuronal degeneration or apoptosis. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as inhibition of cell degeneration or apoptosis, or to enhance synaptic plasticity. Typically, a prophylactic dose is used in subjects prior to or at an earlier stage of disease, so that a prophylactically effective amount may be less than a therapeutically effective amount. A preferred range for therapeutically or prophylactically effective amounts of a compound may be 0.1 nM-0.1M, 0.1 nM-0.05M, 0.05 nM-15 μM or 0.01 nM-10 μM. A pharmacologically effective amount of a medicament refers to using an amount of a medicament present in such a concentration to result in a therapeutic or prophylactic level of drug delivered over the term that the drug is used. This may be dependent on mode of delivery, time period of the dosage, age, weight, general health, sex and diet of the subject receiving the medicament.

Dosage values may vary with the severity of the condition to be alleviated or with the route of administration selected. For example, for oral administration, dosage values may be higher than for intravenous or intraperitoneal administration. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

Compounds of the invention can be provided alone or in combination with other compounds (for example, nucleic acid molecules, small molecules, peptides, or peptide analogues), in the presence of a liposome, an adjuvant, or any pharmaceutically acceptable carrier, in a form suitable for administration to humans. If desired, treatment with a compound according to the invention may be combined with more traditional and existing therapies for neurological damage, synaptic plasticity, learning or memory, or substance abuse. For example, compounds according to the invention may be administered as combination therapy with other treatments such as free-radical inhibitors to maximise neuronal survival; as complementary therapy to anti-coagulant prophylaxis in subjects undergoing atrial fibrillation or are considered to be at risk for stroke. In some embodiments, the compounds may be administered at specific therapeutic windows. For example, in some embodiments, the compounds may be administered approximately 1, 2, 3, 4, 5 or more hours after onset of ischemia.

Disorders or conditions which includes neural dysfunction, for example due to neurological damage or behavioural sensitization due to the excessive activation of NMDA receptors may be treated, prevented, or studied according to alternative embodiments of the methods and compounds of the invention. For example, disorders associated with conditions ranging from hypoglycemia, hypoxia, and cardiac arrest to epilepsy may have components that involve neurological damage disorders according to the invention. Disorders according to the invention include without limitation cerebral ischemia, occurring for example after stroke (ischemic stroke due to for example atherothrombotic disease of e.g., extracranial arteries, or to emboli from the heart or lacunar infarcts) or brain trauma (e.g., intracerebral hemorrhage or subarachnoid hemorrhage); head injury; neurodegenerative disorders in which compromised neurons become sensitive to excitotoxic damage; Alzheimer's disease, Parkinson's disease, Huntington's disease; cognitive impairment associated with schizophrenia; chemotherapy-induced neuropathy; Down's Syndrome; Korsakoff's disease; cerebral palsy; epilepsy; neuropathic pain; amyotrophic lateral sclerosis (ALS); Hutchinson Gilford syndrome; Neuronal cell death associated with diabetes, ataxia, mental retardation, dementias or ischemia, reperfusion, trauma, hemorrhage, infection, or exposure to a toxic substance. Major risk factors for stroke include smoking, diabetes, obesity, and high blood pressure. Accordingly, subjects having any of these conditions or behaviours may be considered as having a disorder according to the invention. In alternative aspects, the invention may involve treating one or more neuronal tissues in a subject, such as a subject having one or more of the foregoing conditions. Neuronal tissues include all tissues that are comprised at least partly of neurons, such as tissues of the peripheral nervous system (PNS) and the central nervous system (CNS), such as brain, white matter, grey matter, spinal cord or ganglia.

As used herein, a subject amendable to treatment may for example be a human, non-human primate, mammal, warm blooded animal, rodent, rat, mouse, cow, horse, pig, sheep, goat, dog, cat, or Aplysia. The subject may for example be a clinical patient, a clinical trial volunteer, or an experimental animal. The subject may be suspected of having or at risk for having neurological damage or neuronal dysfunction, be diagnosed with neurological damage or neuronal dysfunction, or be a control subject that is confirmed to not have neurological damage or neuronal dysfunction, by virtue of diagnostic methods for neurological damage or neuronal dysfunction and the clinical delineation of neurological damage or neuronal dysfunction.

Definitions

“NMDA” is the synthetic amino acid N-methyl-D-aspartate that binds selectively to a subset of glutamate receptors on neurons. These receptors are collectively referred to as NMDA receptors (NMDAR). NMDAR are bound selectively by glutamate, resulting in the opening of calcium channels for neuronal signaling. A ‘synaptic’ receptor or cellular substructure is one found in the area of the synapse in a neuron. An ‘extrasynaptic’ receptor or cellular substructure is one found outside of the area of the synapse in a neuron.

A “neurodegenerative disorder” is a disorder that causes and/or results from degradation of cells of the central nervous system. Various types of neurons or neuronal cells may be involved. Neurodegenerative disorders include Parkinson's disease, Huntington's disease, amyotrophic lateral sclerosis, Alzheimer's disease.

A “chemical entity”, “ligand” or “compound” may include small organic or inorganic molecules with distinct molecular composition made synthetically, found in nature, or of partial synthetic origin. Included in this group are nucleotides, nucleic acids, amino acids, peptides, proteins, or complexes comprising at least one of these entities.

An “agonist” is a chemical entity capable of combining with a receptor on a cell and initiating or enhancing the same reaction or activity otherwise produced by the binding of an endogenous chemical entity.

An “antagonist” is a chemical entity that acts to reduce the physiological activity of another chemical entity, for example by combining with and blocking the receptor of the endogenous chemical entity.

“Cell death” or “apoptosis,” defines a specific execution of programmed cell death that can be triggered by several factors (Krammer et al., 1991). NMDA-mediated neuronal apoptosis is the neuronal cell death observed upon activation of NMDA receptors.

“Modulating” or “modulates” means changing, by either increase or decrease. The increase or decrease may be a change of any value, for example between 10% and 90%, or may be over a threshold value, such as over 10%, 90%, 100%, 200%, 300% or 500% (when compared to a pre-existing or control state).

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

### EXAMPLES

### Methods

Primary Culture of Cortical Neurons

Dissociated cultures of cortical neurons were prepared from 18-day Sprague-Dawley rat embryos as described previously (Mielke et al., 2005). To obtain mixed cortical cultures enriched with neurons, uridine (10 μM) and 5-Fluor-2′-deoxyuridine (10 μM) were added to the culture medium at 3 DIV and maintained for 48 h, to inhibit non-neuronal cell proliferation, before the cultures were shifted back to the normal culture medium. Mature neurons (11-14 DIV) were used for experiments. To induce neuronal apoptosis, cortical cultures were stimulated with NMDA (50 μM) and glycine (10 μM) for 20 min, or STS (100 nM) for 1 h in Mg2+-free extracellular solution (ECS) containing (mM): 25 HEPES acid, 140 NaCl, 33 glucose, 5.4 KCl and 1.3 CaCl2, with pH 7.35 and osmolarity 320-330 mOsm. Specific blockade of synaptic NMDA receptors was achieved by treatment with MK-801 (10 μM) in the presence of bicuculline (50 μM) for 10-15 min in Mg2+-free ECS, followed by thorough wash with ECS containing 1 mM MgCl2 (normal ECS) to remove any trace of MK-801. NR2A-specific antagonist NVP-AAM077 (0.4 μM; generous gift of Y P Auberson, Novartis Pharma AG, Basel, Switzerland) or NR2B-specific antagonist Ro 25-6981 (0.5 μM) was added to the bath medium 10 min prior to and throughout the treatments.

Assessment of Neuronal Apoptosis

To visualize injured neurons, Hoechst-33342 (10 μg/ml) was added to the culture medium 20 h after treatments and incubated for 45 min at 37 oC. Images were taken with a Leica DMIRE2 fluorescence microscope. Quantitative assessment of neuronal apoptosis was performed 20 h following treatments using a Cell Death Detection ELISAPLUS Kit (Roche Applied Science). Absorbance readings were determined using a spectrophotometric microplate reader. Data analyses were carried out according to the manufacturer's instructions. Data are expressed as the difference in apoptosis relative to control and are expressed as a percentage.

Recording of Miniature Excitatory Postsynaptic Currents (mEPSCs) and Whole-cell NMDA Currents

Neurons on coverslips (11 DIV) were transferred to a recording chamber that was continuously perfused with normal ECS. Bicuculline (10 μM) and tetrodotoxin (0.5 μM) were added to isolate action potential-independent miniature excitatory postsynaptic currents (mEPSCs). Patch pipettes were pulled from borosilicate glass capillaries (World Precision Instruments) and filled with an intracellular solution (pH 7.2; 300-310 mOsm) composed of (mM): 140 CsCl gluconate, 0.1 CaCl2, 10 HEPES, 2 MgCl2, 10 BAPTA and 4 ATP. A MultiClamp 700A amplifier (Axon Instruments) was used for the recording. The series resistance was monitored throughout each recording and recordings where the series resistance varied by more than 10% were rejected. No electronic compensation for series resistance was employed. Whole-cell patch-clamp recordings were performed under voltage-clamp mode at a holding membrane potential of −60 mV. Recordings were low-pass filtered at 2 kHz, sampled at 10 kHz, and stored as data files using Clampex 8.0 (Axon). Synaptic events were analyzed offline using the Mini Analysis Program 6.0 (Synaptosoft). During recording, Mg2+-free ECS was used so that mEPSCs comprising both AMPA and NMDA receptor-mediated components could be measured. NMDA receptor antagonists (APV, NVP-AAM077 or Ro 25-6981) were bath applied for at least 10 min to obtain sufficient recording data for analysis after achieving a stable level of NMDA receptor blockade. Synaptic events before and after application of NMDA receptor antagonists were automatically detected from computer stored recordings using the same detection parameters in Mini Analysis Program. Subtraction of averaged traces was done in Excel (Microsoft).

Whole-cell NMDA currents were recorded at a hold membrane potential of −60 mV under voltage-clamped configuration and the currents were evoked by NMDA at concentrations specified in the results in Mg2+-free ECS using a fast perfusion system (Warner Instruments).

Western Blotting

Twelve hours after treatments, proteins were extracted from neurons using a lysis buffer composed of 150 mM NaCl, 50 mM Tris (pH 7.4), 0.1% SDS, 1% NP-40, 0.5% sodium deoxycholate, 1 mM EDTA, 1 mM Na3VO4, 10 μg/ml each of leupeptin and aprotinin, and 1 mM phenylmethylsulfonyl fluoride. To determine the state of Akt phosphorylation, the samples were separated on 10% SDS-PAGE gels, transferred to PVDF membrane and immunoblotted with anti-phosphoSer473-Akt antibody (Cell Signaling). The same membrane was stripped and reprobed with anti-Akt antibody (Cell Signaling). To determine the activity of caspase-3, the samples were separated on 15% SDS-PAGE gels and transferred to PVDF membranes, which were then sequentially probed with antibodies against cleaved caspase-3 (Asp175, Cell Signaling) and β-tubulin (Sigma).

Experimental Stroke In Vitro and In Vivo

OGD was achieved by transferring cortical cultures to an anaerobic chamber (Thermo EC) containing a 5% CO2, 10% H2, and 85% N2 (<0.01% O2) atmosphere (Goldbert et al., 1993; Aarts et al., 2002; Mielke et al., 2005), and then washed 3 times with glucose-free bicarbonate-buffered solution (deoxygenated in the anaerobic chamber for 30 min before use) and maintained anoxic for 1 h at 37° C. OGD was terminated by washing the cultures twice with normal ECS, and then the neurons were switched back to the original growth conditions until further assay.

Transient cerebral focal ischemia was produced by middle cerebral artery occlusion (MCAo) as described (Aarts et al., 2002; Bederson et al., 1986; Longa et al., 1989). Briefly, male Sprague-Dawley rats (Charles River Laboratories) weighing ˜300 g were anesthetized and MCAo was achieved by introducing a 3-0 monofilament suture into the MCA via the internal carotid artery. Body temperature was maintained at 37.0±0.5° C., and blood pressure and blood gases were monitored during the experiments. Animals were sacrificed 24 h following MCAo onset. Cerebral infarction was analyzed using brain sections stained with hematoxylin and eosin (H & E) or 2,3,5-triphenyltetrazolium chloride (TTC). 10 min before the animals were sacrificed, two tests, the postural reflex test to examine upper body posture (Bederson et al., 1986) and the forelimb placing test to examine sensorimotor integration in forelimb placing responses to visual, tactile, and proprioceptive stimuli (De Ryck et al., 1989), were performed to grade neurological function on a scale of 0 to 12 (0=normal, 12=worst). In the pretreatment study, a single bolus of drugs (NR2A-specific antagonist NVP-AAM077 (2.4 mg/kg) or NR2B specific antagonist Ro 25-6981 (6 mg/kg) or vehicle (saline)) was infused intravenously 30 min before a 1-h MCAo. For post-treatment experiments, animals were subjected to a 1.5-h MCAo and drug treatments (glycine, 800 mg/kg; NVP-AAM077 and Ro 25-6981 at the same doses as in the pretreatment study) were then given via intraperitoneal injection (i.p.) 3 h after reperfusion (4.5 h after the onset of MCAo).

Recording of NMDA-induced currents mediated by extrasynaptic NMDA Receptors: Extrasynaptic NMDA receptors were isolated by a specific blockade of synaptic NMDA receptors with NMDA receptor open channel blocker MK-801 as described above. The coverslip with the treated cortical neurons was transferred to a recording chamber for whole-cell patch-clamp recording. Extrasynaptic NMDA receptors in voltage-clamped cortical neurons were activated by NMDA (200 μM) in Mg2+-free ECS using a fast perfusion system (Warner Instruments).

Data Analysis

Data are expressed as mean±SEM. ANOVA was used for comparison among multiple groups, followed by the Holm-Sidak test for comparison between two groups. Statistical significance was defined as p<0.05.

### Example 1

NR2A- and NR2B-containing NMDA Receptors Have Differential Roles in Neuronal Survival.

The roles of NR2A- and NR2B containing NMDA receptors in mediating NMDA-induced neuronal death were established using subunit-specific NMDA receptor antagonists in rat cortical cultures of 11-14 days in vitro (DIV). NMDA receptor-mediated neuronal death was induced by incubating neuronal cultures with 50 μM NMDA plus 10 μM glycine for 20 min (NMDA-mediated excitotoxicity). Neuronal injuries were determined 20 h after treatment by nucleus staining with Hoechst-33342.

NMDA treatment induced neuronal injuries, illustrated by an increase in the proportion of neurons displaying nuclear condensation and/or fragmentation (FIG. 2A). The neuronal apoptosis was confirmed using a quantitative biochemical measurement of intranucleosomal fragmentation (Cell death ELISA) (FIG. 2B). The NMDA-induced neuronal damage was a result of the specific activation of NMDA receptors, as it was fully blocked by the NMDA receptor antagonist, APV (50 μM)

### Example 2

Individual Roles of NR2A- and NR2B-containing NMDA Receptor Subtypes.

To determine the individual roles of NR2A- and NR2B-containing NMDA receptor subtypes in NMDA-induced neuronal apoptosis, subunit-specific 25 antagonists were used to block the receptors. Bath application of Ro 25-6981 (0.5 μM), a specific NR2B-containing receptor antagonist (Mutel et al., 1998), prevented NMDA-induced neuronal apoptosis, indicating the critical involvement of this NMDA receptor subtype. In contrast, application of NR2A subunit-specific antagonist NVP-AAM077 (0.4 μM) (Liu 2004, supra) not only failed to block, but significantly enhanced, NMDA induced apoptosis (FIGS. 2A, B; p<0.05 compared with NMDA alone). These unexpected results indicate that activation of NR2A-containing NMDA receptors exerts a cell survival promoting effect that counteracts the apoptotic action produced by NR2B-containing receptors.

### Example 3

Expression of NR2A- and NR2B-containing NMDA Receptors at Synaptic and Extrasynaptic Sites.

Expression of functional NR2B-containing NMDA receptors at the synaptic site of cultured cortical neurons was examined using whole-cell recording of spontaneous miniature excitatory postsynaptic currents (mEPSCs). mEPSCs were recorded in whole-cell voltage-clamp mode at a holding membrane potential of −60 mV in the presence of tetrodotoxin (0.5 μM) and bicuculline (10 μM) with zero added Mg2+. Under these recording conditions, mEPSCs are comprised of both a fast, α-amino-3-hydroxy-5-methyl-4-isoxazole propionic acid (AMPA) subtype glutamate receptor-mediated component, and a slow, NMDA receptor-mediated component (FIG. 3A). The fast AMPA 5 receptor-mediated component was completely blocked by the non-NMDA receptor antagonist 6,7-dinitroquinoxaline-2,3-dione (DNQX), while the slow NMDA receptor-mediated component was fully blocked by the NMDA receptor antagonist APV (FIGS. 3Aa, Ab, Ad). The NMDA receptor-mediated component was significantly reduced by bath application of the specific 10 NR2B-containing NMDA receptor antagonist Ro 25-6981 (0.5 μM; FIGS. 3Ac, Ad). As mEPSCs are primarily mediated by synaptically localized receptors activated by glutamate spontaneously released from presynaptic terminals, this sensitivity to NR2B antagonist demonstrates that functional NR2B-containing NMDA receptors are present within the glutamatergic synapses of the neurons under study.

On average, the NR2B-containing receptor-mediated component accounted for 32.4±3.6% of the synaptic NMDA currents (n=5; FIGS. 3Ac, Ad) and the remainder was primarily mediated by NR2A-containing receptors as it was largely eliminated in the presence of the NR2A-specific antagonist NVPAAM077 (0.4 μM; n=5). Thus, similar to hippocampal CA1 neurons in brain slices, functional subpopulations of both NR2A- and NR2B-containing NMDA receptors are expressed at the synapses of the cultured neurons. Specifically at the synapse, however, NR2A-containing NMDA receptors dominate.

### Example 4

Function of NR2A- and NR2B Synaptic Receptor Subpopulations in Mediating Neuronal Death.

If the location of the receptors is the determining factor in their activity in mediating neuronal survival or death, activation of either receptor population at the synapse should promote neuronal survival. However, if the subunit composition is the determinant, the two populations will demonstrate opposing actions.

Activation of synaptic NMDA receptors by synaptically released glutamate was increased by incubating neurons with the GABAA receptor antagonist bicuculline (50 μM, 4 h). Bicuculline increases neuronal excitation by blocking the GABAA receptor-mediated synaptic inhibition and thereby enhances action potential-dependent synchronized release of glutamate from presynaptic terminals. Neuronal apoptosis was quantified 20 h following the treatments. Stimulation of synaptic NMDA receptors by application of bicuculline alone, or in the presence of NR2B antagonist Ro 25-6981, did not cause apoptotic cell death (FIG. 3B). In contrast, blocking synaptic NR2A-containing receptors by co-application of NVP-AAM077 with bicuculline significantly increased neuronal apoptosis (p<0.001; FIG. 3B). The NR2A blockade-induced neuronal apoptosis was mediated by synaptic NR2B-containing receptors as it was prevented in the presence of Ro 25-6981 (p<0.01; FIG. 3B). Under bicuculline incubation, the increased action potential-dependent synaptic release of glutamate may lead to activation of extrasynaptic NMDA receptors by glutamate spillover.

The impact of a blockade of synaptic NMDA receptor activation by glutamate spontaneously released from terminals under basal, non-stimulated conditions was subsequently examined. Incubation of neurons with NVP-AAM077 for 4 h failed to increase neuronal apoptosis. However, when the incubation time was increased to 48 h, a significant increase in neuronal apoptosis was observed (FIG. 3C, p<0.01). Synaptic NR2A antagonist-induced apoptosis was also prevented by the blockade of synaptic NR2B receptors with Ro 25-6981. In contrast, blockade of synaptic NR2B alone for up to 48 h did not increase neuronal apoptosis (FIG. 3C). These results demonstrate that both synaptic NR2A- and NR2B-containing receptors are activated by spontaneously released glutamate from the presynaptic terminal and hence tonically exert opposing influences with respect to promoting cell survival or death. Under physiological conditions, the NR2A-mediated cell survival-promoting effect counteracts the tonic apoptotic action of NR2B, thereby maintaining normal neuronal survival. As such, synaptic NR2B-mediated neuronal death can only be unmasked after pharmacological blockade of the NR2A-mediated cell survival signalling pathway. In contrast to the predominant expression of NR2A-containing receptors at synapses, NR2B-containing receptors are thought to be the predominant NMDA receptor expressed at extrasynaptic sites in natural neurons (Tovar, K. supra; Massey et al., 2004).

### Example 5

NR2A-containing Receptors are Under-represented at Extrasynaptic Sites in Natural Neurons.

NMDA receptors expressed at synapses were blocked pharmacologically and subsequently tested as to whether currents gated through extrasynaptic NMDA receptors were sensitive to NR2A subunit-specific antagonism. The selective blockade of synaptic NMDA receptors was achieved by coapplication of bicuculline (50 μM) and MK-801 (10 μM) for 10 min. MK-801, as an irreversible blocker of open

NMDA receptor channels, can only block the bicuculline-activated synaptic NMDA receptors, and cannot block extrasynaptic channels that are not activated during bicuculline application (Huettner et al., 1988). The complete blockade of synaptic NMDA receptors could be achieved within 10 min of bicuculline and MK-801 coapplication as indicated by the virtual elimination of the slow, APV-sensitive component of mEPSCs (FIGS. 4Aa, Ab). Little recovery was observed one hour following wash-out of the drugs. The currents gated through extrasynaptic NMDA receptors were then induced by application of NMDA (200 μM) via a fast perfusion system after washing out bicuculline and MK801. The extrasynaptic NMDA receptor-mediated currents were largely reduced by the NR2B antagonist Ro 25-6981 (FIGS. 4Ac, Ad), consistent with the idea that extra synaptic NMDA receptors are predominantly NR2B containing (Stocca et al., 1998). The residual, NR2B antagonist-resistant current was blocked by the NR2A antagonist NVP-AAM077 (FIGS. 4Ac, Ad), indicating that the non-NR2B-containing extrasynaptic NMDA receptors were NR2A-containing receptors. On average, about 26.6±2.3% (n=5) of total currents gated by extrasynaptic NMDA receptors were mediated by NR2A-containing receptors (FIG. 4Ad). These results provide evidence for the existence of a substantial number of functional extrasynaptic NR2A-containing NMDA receptors in mature cultured cortical neurons.

### Example 5

Role of Extrasynaptic NMDA Receptors in Mediating Cell Survival and Death.

Synaptic NMDA receptors were blocked with bicuculline and MK-801, and the neurons were treated with NMDA (50 μM plus 10 μM glycine) for 20 min in the absence or presence of NVP-AAM077 (0.4 μM) or Ro 25-6981 (0.5 μM).

Quantitative neuronal apoptosis assays performed 20 h after the treatments showed that NMDA application alone (non-selective activation of extrasynaptic NMDA receptors) elicited significant apoptosis (p<0.001, FIG. 4B) which could be prevented by a selective blockade of NR2B-containing extrasynaptic NMDA receptors with Ro 25-6981. In sharp contrast, blockade of the NR2A-containing receptors with NVP-AAM077, i.e. leaving NR2B-containing NMDA receptors intact, did not prevent, but instead potentiated NMDA-mediated apoptosis (p<0.05 compared with NMDA treatment). Thus, as with synaptic NMDA receptors, activation of extrasynaptic NR2A containing receptors has a role in promoting cell survival, counteracting NR2B-containing receptor-mediated neuronal apoptosis. Taken together, the data illustrated in FIGS. 3 and 4 illustrate that, regardless of their anatomical (synaptic vs. extrasynaptic) locations, NR2A-and NR2B9 containing receptors have opposing roles in mediating NMDA-elicited neuronal survival and apoptosis.

### Example 6

NR2A survival Effect Protects Against Non-NMDA Receptor-mediated Neuronal Damage

Following an irreversible blockade of all synaptic NMDA receptors with coapplication of bicuculline and MK-801, and in the presence of Ro 25-6981, bath application of NMDA (200 μM, 5 min) did not increase neuronal apoptosis on its own, confirming the effective blockade of NR2B-containing receptor-mediated apoptotic actions by Ro 25-6981. Staurosporine is a potent kinase inhibitor and inducer of apoptosis, however, the application of NMDA significantly reduced staurosporine (STS)-induced apoptosis (100 nM, 1 h) (p 15<0.001 compared with STS alone; FIG. 5A). The NMDA-induced neuronal protection was mediated by NR2A-containing receptors as it was prevented by co-application of NVP-AAM077 (p<0.001 compared with STS alone). Thus, the NR2A-containing NMDA receptor-mediated pro-survival pathway is able to guard against both NMDA receptor-and non-NMDA receptor mediated neuronal damage.

### Example 7

NR2A- and NR2B-mediation of Cell Survival in an In Vitro Stroke Model.

A well characterized in vitro stroke model, oxygen and glucose deprivation (OGD) (Goldberg, supra; Aarts, 2002, supra) was employed to further examine the opposing roles of NR2A- and NR2B-containing NMDA receptors in mediating cell death. Cortical cultures of 11-14 DIV were exposed to an anaerobic atmosphere for 1 h in a glucose-free solution in the absence or the presence of either NVPAAM077 (0.4 μM) or Ro 25-6981 (0.5 μM). Neuronal apoptosis was quantitatively determined 20 h after OGD. As shown in FIG. 6A, 1 h of OGD was able to produce a pronounced increase in neuronal apoptosis. Selective inhibition of the NR2A-containing NMDA receptors with NVP-AAM077 significantly enhanced OGD-induced neuronal apoptosis (p<0.05 compared with OGD), and in contrast, a specific blockade of the NR2B-containing NMDA receptors by Ro 25-6981 drastically reduced the ODG-induced apoptosis (p<0.001 compared with OGD; FIG. 6A).

### Example 8

NR2A- and NR2B-mediation of Cell Survival in an In Vivo Stroke Model.

The in vitro stroke model experiments were subsequently validated in a rat focal ischemic stroke model—middle cerebral artery occlusion (MCAo) (Bederson et al., 1986). NVPAAM077 (2.4 mg/kg), Ro 25-6981 (6 mg/kg) or vehicle (saline) were infused intravenously in the rats 30 min prior to stroke onset. The animals were then subjected to a 1-h transient ischemic stroke induced by MCAo. This relatively short duration of ischemia was chosen to unmask the potential neuroprotective effects mediated by NR2A-containing receptors activated during the stroke challenge. Neurological score and cerebral infarction were examined 24 h after the MCAo onset. Blockade of NR2A-containing NMDA receptors significantly increased the infarct areas and the total infarct volume, whereas, in sharp contrast, the stroke-induced brain injuries were remarkably reduced by NR2B antagonism (FIGS. 6B, C). Specifically, when compared with saline-treated animals, NVP-AAM077 pre-treatment gave rise to a 67.0±17.9% increase in total infarct volume (n=5; p<0.05), while Ro 25-6981 treatment decreased the total infarct volume by 67.8±4.3% (n=6; p<0.01). Neurological behavioral tests showed that the NVP-AAM077-treated animals exhibited a trend toward poorer neurological function while Ro 25-6981 treatment produced a significant protective effect (FIG. 6D). Together these observations indicate that both NR2A- and NR2B-containing NMDA receptor subtypes are activated during stroke, exerting opposing effects on ischemic brain damage.

The effectiveness of the NR2B-specific antagonist in reducing brain damage is consistent with the hypothesis that a massive increase in extracellular glutamate concentration immediately following stroke activates extrasynaptic NR2B-containing receptors and their downstream neuronal death pathway. However, as the extracellular glutamate concentration rapidly recovers to pre-stroke levels (Benveniste, supra), and extrasynaptic NR2B-containing receptors are not activated thereafter, an NR2B antagonist has a narrow window of efficacy. In contrast, selective activation of NR2A-containing receptors initiates cell survival promoting signals, protecting neurons against ischemic damage irrespective of the time in relation to the stroke event, and have a much broader therapeutic window.

### Example 9

Post-ischemic Potentiation of the Pro-survival Action of NR2A-containing 15 NMDA, is Neuroprotective.

A 1.5-h MCAo challenge was administered to the rats, and pharmacological blockade of NR2B- and/or selective activation of NR2A-containing receptors was achieved by administration of respective drugs intraperitoneally 4.5 h after stroke onset. As shown in FIG. 7, administration of the selective NR2B antagonist Ro 25-6981 (n=10) did not provide any noticeable neuroprotection when compared with MCAo alone (saline injection; n=10). The activating effect on NR2A-containing receptors was mimicked with the application of the NMDA receptor co-agonist glycine in the presence of an 25 NR2B antagonist. Glycine by itself potentiates the function of NMDA receptors that are activated by endogenously released glutamate from presynaptic terminals both in vitro and in vivo (Johnson et al., 1987; Lu et al., 2001; De et al., 2000). Administration of glycine (800 mg/kg; n=9) in combination with Ro 25-6981 4.5 h after the onset of stroke resulted in a significant reduction in total infarct volume assessed 24 h after MCAo onset (54.3±9.2%, p<0.001; FIG. 7). Glycine in combination with Ro 25-6981 also improved neurological function scores tested 24 h following stroke onset (p<0.001; FIG. 7). Glycine in combination with Ro 25-6981 specifically enhances NR2A-containing NMDA receptor-mediated cell survival promoting action. This enhancement of cell survival is abolished by co-administration of the NR2A-specific antagonist NVP-AAM077 in combination with glycine (n=10; FIG. 7).

### REFERENCES

The following documents are incorporated herein by reference, as if each individual publication were specifically and individually indicated to be incorporated by reference herein and as though fully set forth herein.
Aarts, M., Iihara, K., Wei, W. L., Xiong, Z. G., Arundine, M., Cerwinski, W., MacDonald, J. F., and Tymianski, M. 2003. A key role for TRPM7 channels in anoxic neuronal death. Cell 115:863-877.
Aarts, M., Liu, Y., Liu, L., Besshoh, S., Arundine, M., Gurd, J. W., Wang, Y. T., Salter, M. W., and Tymianski, M. 2002. Treatment of ischemic brain damage by perturbing NMDA receptor-PSD-95 protein interactions. Science 298:846-850.
Alanine et al. 2003. 1-Benzyloxy-4,5-dihydro-1H-imidazol-2-yl-amines, a novel class of NR1/2B subtype selective NMDA receptor antagonists. Bioorg. Med. Chem. Lett. 13(19): 3155-59
Albensi, B. C., Igoechi, C., Janigro, D., and Ilkanich, E. 2004. Why do many NMDA antagonists fail, while others are safe and effective at blocking excitotoxicity associated with dementia and acute injury? Am. J. Alzheimers. Dis. Other Demen. 19:269-274.
Amadoro, G., Ciotti, M. T., Costanzi, M., Cestari, V., Calissano, P., and Canu, N. Feb. 21, 2006. NMDA receptor mediates tau-induced neurotoxicity by calpain and ERK/MAPK activation. Proc Natl Acad Sci USA. 103(8):2892-7
Arundine, M. and Tymianski, M. 2004. Molecular mechanisms of glutamate-dependent neurodegeneration in ischemia and traumatic brain injury. Cell Mol. Life Sci. 61:657-668
Arundine, M., and Tymianski, M. 2004. Molecular mechanisms of glutamate-dependent neurodegeneration in ischemia and traumatic brain injury. Cell Mol. Life Sci. 61:657-668.
Avenet et al. 1997. Antagonist properties of eliprodil and other NMDA receptor antagonists at rat NR1A/NR2A and NR1A/NR2B receptors expressed in Xenopus oocytes. Neurosci. Lett. 223(2): 133-36
Bayer et al. Jan. 25, 2006. Transition from reversible to persistent binding of CaMKII to postsynaptic sites and NR2B. J Neurosci. 26(4):1164-74
Bederson, J. B. et al. 1986. Rat middle cerebral artery occlusion: evaluation of the model and development of a neurologic examination. Stroke. 17:472-476
Bederson, J. B., Pitts, L. H., Tsuji, M., Nishimura, M. C., Davis, R. L., and Bartkowski, H. 1986. Rat middle cerebral artery occlusion: evaluation of the model and development of a neurologic examination. Stroke 17:472-476.
Benveniste, H., Drejer, J., Schousboe, A., and Diemer, N. H. 1984. Elevation of the extracellular concentrations of glutamate and aspartate in rat hippocampus during transient cerebral ischemia monitored by intracerebral microdialysis. J. Neurochem. 43:1369-1374.
Berberich, S., Punnakkal, P., Jensen, V., Pawlak, V., Seeburg, P. H., Hvalby, O., and Kohr, G. 2005. Lack of NMDA receptor subtype selectivity for hippocampal long-term potentiation. J Neurosci. 25:6907-6910.
Blaise, M-C, Sowdhamini, R., Rao, M. R. P., and Pradhan, N. December 2004. Evolutionary trace analysis of ionotropic glutamate receptor sequences and modeling the interactions of agonists with different NMDA receptor subunits. Journal of Molecular Modeling. 10(5-6)305-316

Blanchet, P. J., Konitsiotis, S., Whittemore, E. R., Zhou, Z. L., Woodward, R. M. and Chase, T. N. Sep. 1, 1999. Differing Effects of N-methyl-D-aspartate Receptor Subtype Selective Antagonists on Dyskinesias in Levodopa-Treated 1-Methyl-4-phenyl-tetrahydropyridine Monkeys. J. Pharmacol. Exp. Ther. 290(3):1034-1040
Bliss, T., and Schoepfer, R. 2004. Neuroscience. Controlling the ups and downs of synaptic strength. Science 304:973-974.
Boje and Lakhman. 1998. Chronic dosing with 1-aminocyclopropoanecarboxylic acid, a glycine partial agonist, modulates NMDA inhibition of muscarinic-coupled PI hydrolysis in rat cortical slices. Neurochem Res. 23(9): 1167-74
Boyce-Rustay and Cunningham. 2004. The role of NMDA receptor binding sites in ethanol place conditioning. Behav. Neurosci. 118(4): 822-34
Bresink et al. 1996. Effects of memantine on recombinant rat NMDA receptors expressed in HEK 293 cells. Br. J. Pharmacol. 119(2): 195-204
Brimecombe, J. C., Gallagher, M. J., Lynch, D. R., and Aizenman, E. Aug. 1, 1998. An NR2B Point Mutation Affecting Haloperidol and CP101,606 Sensitivity of Single Recombinant N-Methyl-D-Aspartate Receptors. J. Pharmacol. Exp. Ther. 286(2):627-634
Budd, S. L., and Lipton, S. A. 1999. Signaling events in NMDA receptor-induced apoptosis in cerebrocortical cultures. Ann. N. Y. Acad. Sci. 893:261-264.
Budd, S. L., Tenneti, L., Lishnak, T., and Lipton, S. A. 2000. Mitochondrial and extramitochondrial apoptotic signaling pathways in cerebrocortical neurons. Proc. Natl. Acad. Sci. U.S.A 97:6161-6166.
Buller, A. L., Larson, H. C., Schneider, B. E., Beaton, J. A., Morrisett, R. A. and Monaghan, D. T. 1994. The molecular basis of NMDA receptor subtypes: native receptor diversity is predicted by subunit composition. Journal of Neuroscience, Vol 14:5471-5484
Butler et al. 1998. (3R,4S)-3-[4-(4-fluorophenyl)-4-hydroxypiperidin-1-yl]chroman-4,7-diol: a conformationally restricted analogue of the NR2B subtype-selective NMDA antagonist (1S,2S)-1-(4-hydroxyphenyl)-2-(4-hydroxy-4-phenylpiperidino)-1-propanol. J. Med. Chem. 41(7): 1172-84
Ceccona, M., Rumbaughb, G. and Vicini, S. March 2001. Distinct effect of pregnenolone sulfate on NMDA receptor subtypes. Neuropharmacology. 40(4)491-500
Chalecka-Franaszek, E., and Chuang, D. M. 1999. Lithium activates the serine/threonine kinase Akt-1 and suppresses glutamate-induced inhibition of Akt-1 activity in neurons. Proc. Natl. Acad. Sci. U.S.A 96:8745-8750.
Chazot, P. L. 2000. CP-101606 Pfizer Inc. Curr. Opin. Investig. Drugs. 1:370-374
Chazot, P. L. 2000. CP-101606 Pfizer Inc. Curr. Opin. Investig. Drugs 1:370-374.
Chazot. 2000. CP-101606. Curr. Opin. Investig. Drugs 1(3): 370-74
Chenard and Menniti. 1999. Antagonists selective for NMDA receptors containing the NR2B subunit. Curr. Pharm. Des. 5(5): 381-404
Choi, D. W. 1994. Calcium and excitotoxic neuronal injury. [Review]. Annals of the New York Academy of Sciences 747:162-171.
Christie, J. M., Jane, D. E. and Monaghan, D. T. Mar. 1, 2000. Native N-Methyl-D-aspartate Receptors Containing NR2A and NR2B Subunits Have Pharmacologically Distinct Competitive Antagonist Binding Sites. J. Pharmacol. Exp. Ther. 292(3):1169-1174
Chu et al. 1995. Ethanol inhibition of recombinant heteromeric NMDA channels in the presence and absence of modulators. J. Neurochem. 65(1):140-48
Coffer, P. J., Jin, J., and Woodgett, J. R. 1998. Protein kinase B (c-Akt): a multifunctional mediator of phosphatidylinositol 3-kinase activation. Biochem. J. 335 (Pt 1):1-13.
Conantokin G; Thominiaux et al. 2006. Radiosynthesis of (E)-N-(2-[11C]methoxybenzyl)-3-phenyl-acrylamidine, a novel subnanomolar NR2B subtype-selective NMDA receptor antagonist. Appl. Radiat. Isot. 64(3): 348-54
Corbett, D., and Nurse, S. 1998. The problem of assessing effective neuroprotection in experimental cerebral ischemia. Prog. Neurobiol. 54:531-548.
Cull-Candy, S. G., and Leszkiewicz, D. N. 2004. Role of distinct NMDA receptor subtypes at central synapses. Sci. STKE. 2004:re16.
Danton and Dietrich. 2004. The Search for Neuroprotective Strategies in Stroke. American Journal of Neuroradiology. 25(2):181-194
Danysz and Parsons. 1998. Glycine and N-Methyl-D-Aspartate Receptors: Physiological Significance and Possible Therapeutic Applications. Pharmacological Reviews 50(4):597
De Ryck, M., Van, R. J., Borgers, M., Wauquier, A., and Janssen, P. A. 1989. Photochemical stroke model: flunarizine prevents sensorimotor deficits after neocortical infarcts in rats. Stroke 20:1383-1390.
De, S. G., et al. 2000. NMDA and AMPA/kainate receptors are involved in the anticonvulsant activity of riluzole in DBA/2 mice. Eur. J. Pharmacol. 408:25-34
De, S. G., Siniscalchi, A., Ferreri, G., Gallelli, L., and De, S. A. 2000. NMDA and AMPA/kainate receptors are involved in the anticonvulsant activity of riluzole in DBA/2 mice. Eur. J. Pharmacol. 408:25-34.
Delkara et al., 1990
Di et al. 1997. Effect of CP101,606, a novel NR2B subunit antagonist of the N-methyl-D-aspartate receptor, on the volume of ischemic brain damage off cytotoxic brain edema after middle cerebral artery occlusion in the feline brain. Stroke 28(11):2244-51
Dingledine. R., Borges, K., Bowie, D. and Traynelis, S. F. March 1999. The Glutamate Receptor Ion Channels Pharmacological Reviews. Vol. 51, Issue 1:7-62
Donevan and McCabe. 2000. Conantokin G is an NR2B-selective competitive antagonist of N-methyl-D-aspartate receptors. Mol. Pharmacol. 58(3):614-23
Donevan, S. D. and McCabe, R. T. Sep. 1, 2000. Conantokin G Is an NR2B-Selective Competitive Antagonist of N-Methyl-D-aspartate Receptors Mol. Pharmacol. 58(3):614-623
Dudek, H., Datta, S. R., Franke, T. F., Birnbaum, M. J., Yao, R., Cooper, G. M., Segal, R. A., Kaplan, D. R., and Greenberg, M. E. 1997. Regulation of neuronal survival by the serine-threonine protein kinase Akt. Science 275:661-665.
Fischer, G., Mutel, V., Trube, G., Malherbe, P., Kew, J. N., Mohacsi, E., Heitz, M. P., and Kemp, J. A. 1997. Ro 25-6981, a highly potent and selective blocker of N-methyl-D-aspartate receptors containing the NR2B subunit. Characterization in vitro. J Pharmacol. Exp. Ther. 283:1285-1292.
Foucaud, B., Laube, B., Schemm, R., Kreimeyer, A., Goeldner, M., and Betz, H. Jun. 27, 2003. Structural Model of the N-Methyl-D-aspartate Receptor Glycine Site Probed by Site-directed Chemical Coupling. J. Biol. Chem. 278(26):24011-24017
Gallagher et al. 1996. Interactions between ifenprodil and the NR2B subunit of the N-methyl-D-aspartate receptor
Gill et al. 2002. Pharmacological characterization of Ro 63-1908 (1-[2-(4-hydroxy-phenoxy)-ethyl]-4-(4-methyl-benzyl)-piperidin-4-ol), a novel subtype-selective N-methyl-D-aspartate antagonist. J. Pharmacol. Exp. Ther. 302(3):940-48
Gill, R., Alanine, A., Bourson, A., Buttelmann, B., Fischer, G., Heitz, M-P, Kew, J. N. C., Levet-Trafit, B., Lorez, H-P, Malherbe, P., Miss, M. T., Mutel, V., Pinard, E., Roever, S., Schmitt, M., Trube, G., Wybrecht, R., Wyler, R., and Kemp, J. A. Sep. 1, 2002. Pharmacological Characterization of Ro 63-1908 (1-[2-(4-Hydroxy-phenoxy)-ethyl]-4-(4-methyl-benzyl)-piperidin-4-ol), a Novel Subtype-Selective N-Methyl-D-Aspartate Antagonist. J. Pharmacol. Exp. Ther., 302(3):940-948
Gladstone, D. J., Black, S. E., and Hakim, A. M. 2002. Toward wisdom from failure: lessons from neuroprotective stroke trials and new therapeutic directions. Stroke 33:2123-2136.

Goldberg, M. P., and Choi, D. W. 1993. Combined oxygen and glucose deprivation in cortical cell culture: Calcium-dependent and calcium-independent mechanisms of neuronal injury. J. Neurosci. 13:3510-3524.
Graham, S. H. and Chen, J. 2001. J. Cereb. Blood Flow Metab 21:99-109
Grimwood et al. 1996. Generation and characterization of stable cell lines expressing recombinant human N-methyl-D-aspartate receptor subtypes. J. Neurochem. 66(6): 2239-47
Guarneri, P., Russo, D., Cascio, C., De Leo, G., Piccoli, T., Sciuto, V., Piccoli, F., and Guarneri, R. 1998. Pregnenolone sulfate modulates NMDA receptors, inducing and potentiating acute excitotoxicity in isolated retina. Journal of Neuroscience Research. 54(6):787-797
Guseva, E. I., Skvortsovaa, V. I., Dambinovac, S. A., Raevskiyb, K. S., Alekseeva, A. A., Bashkatovab, V. G., Kovalenkoa, A. V., Kudrinb, V. S., and Yakovlevaa, E. V. 2000. Neuroprotective Effects of Glycine for Therapy of Acute Ischaemic Stroke. Cerebrovascular Diseases. 10:49-60
Hamilton, N. M. October 2001. Therapeutic potential of steroids for CNS disorders. Expert Opinion on Therapeutic Patents. 11(10)1523-1531
Hammerland et al. 1992. Conantokin-G selectively inhibits N-methyl-D-aspartate-induced currents in Xenopus oocytes injected with mouse brain mRNA. Eur. J. Pharmacol. 226(3):239-44
Hardingham, G. E. et al. 2002. Extrasynaptic NMDARs oppose synaptic NMDARs by triggering CREB shut-off and cell death pathways. Nat. Neurosci.
Hardingham, G. E., Fukunaga, Y. and Bading, H. 2002. Nat. Neurosci.
Hardingham, G. E., Fukunaga, Y., and Bading, H. 2002. Extrasynaptic NMDARs oppose synaptic NMDARs by triggering CREB shut-off and cell death pathways. Nat. Neurosci.405-414.
Harty and Rogawski. 2000. Felbamate block of recombinant N-methyl-D-aspartate receptors: selectivity for the NR2B subunit. Epilepsy Res. 39(1):47-55
Honer, M., Benke, D., Laube, B., Kuhse, J., Heckendorn, R., Allgeier, H., Angst, C., Monyer, H., Seeburg, P. H., Betz, H., and Mohler, H. May 1, 1998. Differentiation of Glycine Antagonist Sites of N-Methyl-D-aspartate Receptor Subtypes. Preferential Interaction of CGP 61594 with NR1/2B Receptors. J. Biol. Chem. 273(18):11158-11163
Hood et al. 1989. D-cycloserine: a ligand for the N-methyl-D-aspartate coupled glycine receptor has partial agonist characteristics. Neurosci. Leett. 98(1): 91-95
Hoyte L., Barber P. A., Buchan A. M., Hill. March 2004. The Rise and Fall of NMDA Antagonists for Ischemic Stroke. Current Molecular Medicine. 4(2):131-136(6)
Huettner, J. E. and Bean, B. P. 1988. Block of N-methyl-D-aspartate-activated current by the anticonvulsant MK-801: selective binding to open channels. Proc. Natl. Acad. Sci. U.S.A 85, 1307-1311
Huettner, J. E., and Bean, B. P. 1988. Block of N-methyl-D-aspartate-activated current by the anticonvulsant MK-801: selective binding to open channels. Proc. Natl. Acad. Sci. U.S.A 85:1307-1311.
Ikonomidou, C. et al. 1999 Blockade of NMDA receptors and apoptotic neurodegeneration in the developing brain. Science 283:70-74
Ikonomidou, C., and Turski, L. 2002. Why did NMDA receptor antagonists fail clinical trials for stroke and traumatic brain injury? Lancet Neurol. 1:383-386.
Ikonomidou, C., Bosch, F., Miksa, M., Bittigau, P., Vockler, J., Dikranian, K., Tenkova, T. I., Stefovska, V., Turski, L., and Olney, J. W. 1999. Blockade of NMDA receptors and apoptotic neurodegeneration in the developing brain. Science 283:70-74.
Johnson, J. W. and Ascher, P. 1987. Glycine potentiates the NMDA response in cultured mouse brain neurons. Nature. 325:529-531
Kemp and McKernan. 2002. NMDA receptor pathways as drug targets. Nature Neuroscience 5:1039-1042
Kemp, J. A., and McKernan, R. M. 2002. NMDA receptor pathways as drug targets. Nat. Neurosci. 5 Supp1:1039-1042.
Kew et al. 1998. State-dependent NMDA receptor antagonism by Ro 8-4304, a novel NR2B selective, non-competitive voltage-independent antagonist. Br. J. Pharmacol. 123(3): 463-72
Kim, M. J., Dunah, A. W., Wang, Y. T., and Sheng, M. Jun. 2, 2005. Differential roles of NR2A- and NR2B-containing NMDA receptors in Ras-ERK signaling and AMPA receptor trafficking. Neuron. 46(5):745-60
Kim, M. J., Dunah, A. W., Wang, Y. T., and Sheng, M. 2005. Differential roles of NR2A- and NR2B-containing NMDA receptors in Ras-ERK signaling and AMPA receptor trafficking. Neuron 46:745-760.
Kinarsky, L., Feng, B., Skifter, D. A., Morley, R. M., Sherman, S., Jane, D. E. and Monaghan, D. T. Jun. 10, 2005. Identification of Subunit- and Antagonist-Specific Amino Acid Residues in the N-Methyl-D-aspartate Receptor Glutamate-Binding Pocket. J. Pharmacol. Exp. Ther. 313(3):1066-1074;
Kirson, E. D. and Yaari, Y. 1996. Synaptic NMDA receptors in developing mouse hippocampal neurons: functional properties and sensitivity to ifenprodil. J Physiol 497:437
Kleckner, N. S., Glazewski, J. C., Chen, C. C., and Moscrip, T. D. May 1, 1999. Subtype-Selective Antagonism of N-Methyl-D-Aspartate Receptors by Felbamate: Insights into the Mechanism of Action. J. Pharmacol. Exp. Ther. 289(2):886-894
Klein, R. C., Prorok, M., Galdzicki, A., and Castellino, F. J. Jul. 20, 2001. The Amino Acid Residue at Sequence Position 5 in the Conantokin Peptides Partially Governs Subunit-selective Antagonism of Recombinant N-Methyl-D-aspartate Receptors. J. Biol. Chem. 276(29):26860-26867
Kohr, G., Jensen, V., Koester, H. J., Mihaljevic, A. L., Utvik, J. K., Kvello, A., Ottersen, O. P., Seeburg, P. H., Sprengel, R., and Hvalby, O. 2003. Intracellular domains of NMDA receptor subtypes are determinants for long-term potentiation induction. J. Neurosci. 23:10791-10799.
Krammer et al. 1991. Apoptosis in the APO-1 System. Apoptosis: The Molecular Basis of Cell Death. Cold Spring Harbor Laboratory Press. 87-99
Krystal, J. H., D'Souza, D. C., Petrakis, I. L., Belger, A., Berman, R. M., Charney, D. S., Abi-Saab, W., Madonick, S. 1999. NMDA Agonists and Antagonists as Probes of Glutamatergic Dysfunction and Pharmacotherapies in Neuropsychiatric Disorders. Harvard Review of Psychiatry. 7(3)125-143
Lanza et al. 1997. Characterization of a novel putative cognition enhancer mediating facilitation of glycine effect on strychnine-resistant sites coupled to NMDA receptor complex. Neuropharmacology 36: 1057-64
Lee, F. J., Xue, S., Pei, L., Vukusic, B., Chery, N., Wang, Y., Wang, Y. T., Niznik, H. B., Yu, X. M., and Liu, F. 2002. Dual regulation of NMDA receptor functions by direct protein-protein interactions with the dopamine D1 receptor. Cell 111:219-230.
Lee, J. M., et al. 1999. The changing landscape of ischaemic brain injury mechanisms. Nature 399:A7-14
Lee, J. M., Zipfel, G. J., and Choi, D. W. 1999. The changing landscape of ischaemic brain injury mechanisms. Nature 399:A7-14.
Lees, K. R., Asplund, K., Carolei, A., Davis, S. M., Diener, H. C., Kaste, M., Orgogozo, J. M., Whitehead, J. Jun. 2, 2000. Glycine antagonist (gavestinel) in neuroprotection (GAIN International) in patients with acute stroke: a randomised controlled trial. GAIN International Investigators. Lancet. 355(9219):1949-54
Li, S., Tian, X., Hartley, D. M., and Feig, L. A. 2006. Distinct roles for Ras-guanine nucleotide-releasing factor 1 (Ras-GRF1) and Ras-GRF2 in the induction of long-term potentiation and long-term depression. J Neurosci. 26:1721-1729.
Lipton, S. A., and Nicotera, P. 1998. Calcium, free radicals and excitotoxins in neuronal apoptosis. Cell Calcium 23:165-171.
Lipton, S. A., and Rosenberg, P. A. 1994. Mechanisms of disease: Excitatory amino acids as a final common pathway for neurologic disorders. N. Engl. J. Med. 330:613-622.
Liu, L., Wong, T. P., Pozza, M. F., Lingenhoehl, K., Wang, Y., Sheng, M., Auberson, Y. P., and Wang, Y. T. 2004. Role of NMDA receptor subtypes in governing the direction of hippocampal synaptic plasticity. Science 304:1021-1024.
Liu, L., Wong, T. P., Pozza, M. F., Lingenhoehl, K., Wang, Y., Sheng, M., Auberson, Y. P., and Wang, Y. T. 2004. Role of NMDA receptor subtypes in governing the direction of hippocampal synaptic plasticity. Science 304:1021-1024
Longa, E. Z., Weinstein, P. R., Carlson, S., and Cummins, R. 1989. Reversible middle cerebral artery occlusion without craniectomy in rats. Stroke 20:84-91.
Loschmann, P. A. et. al. 2004. Exp. Neurol. 187:86-93
Losi et al. 2006. Functional in vitro characterization of CR 3394: a novel voltage dependent N-methyl-D-aspartate (NMDA) receptor antagonist. Neuropharmacology 50(3): 277-85
Losi et al. 2006. Functional in vitro characterization of CR 3394: a novel voltage dependent N-methyl-D-aspartate (NMDA) receptor antagonist. Neuropharmacology 50(3): 277-85
Losi et al. 2006. Functional in vitro characterization of CR 3394: A novel voltage dependent N-methyl-D-asparate (NMDA) receptor antagonist. Neuropharmacology 50: 277-85
Lozovaya et al. 2004. Extrasynaptic NR2B and NR2D subunits of NMDA receptors shape ‘superslow’ afterburst EPSC in rat hippocampus. J. Physiol. 558(Pt 2):451-63
Lu, W. et al. 2001. Activation of synaptic NMDA receptors induces membrane insertion of new AMPA receptors and LTP in cultured hippocampal neurons. Neuron. 29243-254
Lu, W., Man, H., Ju, W., Trimble, W. S., MacDonald, J. F., and Wang, Y. T. 2001. Activation of synaptic NMDA receptors induces membrane insertion of new AMPA receptors and LTP in cultured hippocampal neurons. Neuron 29:243-254.
Malayev, A., Gibbs, T. T., and Farb, D. H. 2002. Inhibition of the NMDA response by pregnenolone sulphate reveals subtype selective modulation of NMDA receptors by sulphated steroids. British Journal of Pharmacology. 135(901-909)
Man, H. Y., Wang, Q., Lu, W. Y., Ju, W., Ahmadian, G., Liu, L., D'Souza, S., Wong, T. P., Taghibiglou, C., Lu, J. et al 2003. Activation of PI3-Kinase Is Required for AMPA Receptor Insertion during LTP of mEPSCs in Cultured Hippocampal Neurons. Neuron 38:611-624.
Margaill, I et al. 1996. J Cereb Blood Flow Metab. 16:107-113
Massey, P. V. et al. 2004. Differential roles of NR2A and NR2B-containing NMDA receptors in cortical long-term potentiation and long-term depression. J. Neurosci. 24, 7821-7828
Massey, P. V., Johnson, B. E., Moult, P. R., Auberson, Y. P., Brown, M. W., Molnar, E., Collingridge, G. L., and Bashir, Z. I. 2004. Differential roles of NR2A and NR2B-containing NMDA receptors in cortical long-term potentiation and long-term depression. J. Neurosci. 24:7821-7828.
Mattson, M. P. 2000. Nat. Rev. Mol. Cell Biol. 1:120-129
Mattson, M. P. 1997. Neuroprotective signal transduction: relevance to stroke [In Process Citation]. Neurosci. Biobehav. Rev. 21:193-206.
Mayer et al. 1987. Agonist- and voltage-gates calcium entry in cultured mouse spinal cord neurons under voltage clamp measured using arsenazo III. J. Neurosci. 7(10): 3230-44
Mayer et al. 1989. Regulation of NMDA receptor desensitization in mouse hippocampal neurons by glycine. Nature 338(6214): 425-27
McBain, C. J., and Mayer, M. L. 1994. N-methyl-D-aspartic acid receptor structure and function. Physiol Rev. 74:723-760.
McCauley, J. A. April 2005. NR2B subtype-selective NMDA receptor antagonists: 2001-2004. Expert Opinion on Therapeutic Patents. 15(4)389-407
Mielke, J. G., and Wang, Y. T. 2005. Insulin exerts neuroprotection by counteracting the decrease in cell-surface GABA receptors following oxygen-glucose deprivation in cultured cortical neurons. J. Neurochem. 92:103-113.
Millan and Seguin. 1993. (+)-HA 966, a partial agonist at the glycine site coupled to NMDA receptors, blocks formalin-induced pain in mice. Eur. J. Pharmacol. 238(2-3): 445-47
Monyer, H., Burnashev, N., Laurie, D. J., Sakmann, B., and Seeburg, P. H. 1994. Developmental and regional expression in the rat brain and functional properties of four NMDA receptors. Neuron 12:529-540.
Mutel et al. 1998. In vitro binding properties in rat brain of [3H]Ro 2506981, a potent and selective antagonist of NMDA receptors containing NR2B subunits. J. Neurochem. 70(5):2147-55
Mutel, V. et al. 1998. In vitro binding properties in rat brain of [3H]Ro 25-6981, a potent and selective antagonist of NMDA receptors containing NR2B subunits. J. Neurochem. 70, 2147-2155
Mutel, V., Buchy, D., Klingelschmidt, A., Messer, J., Bleuel, Z., Kemp, J. A., and Richards, J. G. 1998. In vitro binding properties in rat brain of [3H]Ro 25-6981, a potent and selective antagonist of NMDA receptors containing NR2B subunits. J. Neurochem. 70:2147-2155.
Nagy et al. 2004. NR2B subunit selective NMDA antagonists inhibit neurotoxic effect of alcohol-withdrawal in primary cultures of rat cortical neurones. Neurochem. Int. 44(1): 17-23
Nagy et al. 2004. NR2B subunit selective NMDA antagonists inhibit neurotoxic effect of alcohol-withdrawal in primary cultures of rat cortical neurones. Neurochem. Int. 44(1): 17-23
Nagy et al. 2004. NR2B subunit selective NMDA antagonists inhibit neurotoxic effect of alcohol-withdrawal in primary cultures of rat cortical neurones. Neurochem. Int. 44(1): 17-23
Nagy et al. 2004. NR2B subunit selective NMDA antagonists inhibit neurotoxic effect of alcohol-withdrawal in primary cultures of rat cortical neurones. Neurochem. Int. 44(1): 17-23
Nagy, J., Horvath, C., Farkas, S., Kolok, S., and Szombathelyi Z. January 2004. NR2B subunit selective NMDA antagonists inhibit neurotoxic effect of alcohol-withdrawal in primary cultures of rat cortical neurones. Neurochem Int. 44(1):17-23
Nicotera, P. and Lipton, S. A. 1999. J. Cereb. Blood Flow Metab. 19:583-591
O'Donnell, L. A., Agrawal, A., Jordan-Sciutto, K. L., Dichter, M. A., Lynch, D. R., and Kolson, D. L. 2006. Human immunodeficiency virus (HIV)-induced neurotoxicity: roles for the NMDA receptor subtypes. J Neurosci. 26:981-990.
Okamoto, S. S., Li, Z., Ju, C., Scholzke, M. N., Mathews, E., Cui, J., Salvesen, G. S., Bossy-Wetzel, E., and Lipton, S. A. 2002. Dominant-interfering forms of MEF2 generated by caspase cleavage contribute to NMDA-induced neuronal apoptosis. Proc. Natl. Acad. Sci. U.S.A 99:3974-3979.
Park-Chung, M., Wu, F. S., Purdy, R. H., Malayev, A. A., Gibbs, T. T., Farb, D. H. December 1997. Distinct sites for inverse modulation of N-methyl-D-aspartate receptors by sulfated steroids. Mol Pharmacol. 52(6):1113-23
Parsons, C. G., Danysz, W., and Quack, G. (1999) Memantine is a clinically well tolerated N-methyl-D-aspartate (NMDA) receptor antagonist—a review of preclinical data Neuropharmacology 38: 735-767.
Posey, D. J. et al. 2004. A pilot study of D-cycloserine in subjects with autistic disorder. Am. J. Psychiatry. 161:2115-2117
Posey, D. J., Kern, D. L., Swiezy, N. B., Sweeten, T. L., Wiegand, R. E., and McDougle, C. J. 2004. A pilot study of D-cycloserine in subjects with autistic disorder. Am. J. Psychiatry 161:2115-2117.
Priestley et al. 1995. Pharmacological properties of recombinant N-methyl-D-aspartate receptors comprising NR1a/NR2A and NR1a/NR2B subunit assemblies expressed in permanently transfected mouse fibroblast cells. Mol. Pharmacol. 48(5):841-48
Priestley et al. 1995. Pharmacological properties of recombinant N-methyl-D-aspartate receptors comprising NR1a/NR2A and NR1a/NR2B subunit assemblies expressed in permanently transfected mouse fibroblast cells. Mol. Pharmacol. 48(5): 841-48
Priestley et al. 1995. Pharmacological properties of recombinant N-methyl-D-aspartate receptors comprising NR1a/NR2A and NR1a/NR2B subunit assemblies expressed in permanently transfected mouse fibroblast cells. Mol. Pharmacol. 48(5): 841-48
Priestley et al. 1995. Pharmacological properties of recombinant N-methyl-D-aspartate receptors comprising NR1a/NR2A and NR1a/NR2B subunit assemblies expressed in permanently transfected mouse fibroblast cells. Mol. Pharmacol. 48(5): 841-48
Priestley et al. 1995. Pharmacological properties of recombinant N-methyl-D-aspartate receptors comprising NR1a/NR2A and NR1a/NR2B subunit assemblies expressed in permanently transfected mouse fibroblast cells. Mol. Pharmacol. 48(5): 841-48
Reggiani et al. 1989. Effect of 7-chloro kynurenic acid on glycine modulation of the N-methyl-D-aspartate response in guine-pig myenteric plexus. Eur. J. Pharmacol. 168(1): 123-27
Remington's Pharmaceutical Sciences (19th edition). ed. A. Gennaro. 1995. Mack Publishing Company. Easton, Pa.
Rock, D. M. and R L Macdonald. (April 1995) Polyamine Regulation of N-Methyl-D-Aspartate Receptor Channels. Annual Review of Pharmacology and Toxicology. Vol. 35: 463-482.
Roesler, R., Quevedo, J., Schroder, N. January 2003. Is it time to conclude that NMDA antagonists have failed? Lancet Neurol. 2(1):13
Sacco, R. L., DeRosa, J. T., Haley, E. C., Jr, Levin, B., Ordronneau, P., Phillips, S. J., Rundek, T., Snipes, R. G., and Thompson, J. L. Apr. 4, 2001. Glycine antagonist in neuroprotection for patients with acute stroke: GAIN Americas: a randomized controlled trial. JAMA. 285(13):1719-28
Schoepp et al. 1994. The NMDA receptor agonist DL-(tetraziol-5-yl)glycine is a highly potent excitotoxin. Eur. J. Pharmacol. 270(1): 67-72
Sheinin et al. 2002. Specificity of putative partial agonist, 1-aminocyclopropanecarboxylic acid, for rat N-methyl-D-aspartate receptor subunits. Neurosci. Lett. 317(2): 77-80
Sheng, M. et al., 1994. Changing subunit composition of heteromeric NMDA receptors during development of rat cortex. Nature 368: 144

Sheng, M., and Pak, D. T. 2000. Ligand-gated ion channel interactions with cytoskeletal and signaling proteins. Annu. Rev. Physiol 62:755-78.:755-778.
Singh et al. 1990. Modulation of seizure susceptibility in the mouse by the strychnine-insensitive glycine recognition site of the NMDA receptor/ion channel complex. Br. J. Pharmacol. 99(2): 285-88
Stern et al. 1992. Single-channel conductances of NMDA receptors expressed from cloned cDNAs: comparison with native receptors. Proc. Biol. Sci. 250(1329): 271-77
Stocca, G. and Vicini, S. 1998. Increased contribution of NR2A subunit to synaptic NMDA receptors in developing rat cortical neurons. J. Physiol. 507:13-24
Stocca, G., and Vicini, S. 1998. Increased contribution of NR2A subunit to synaptic NMDA receptors in developing rat cortical neurons. J. Physiol 507:13-24.
Suetake-Koga et al. 2006. In vitro and antinociceptive profile of HON0001, an orally active NMDA receptor NR2B subunit antagonist. Pharmacol. Biochem. Behav.
Thomas, C. G., Miller, A. J., and Westbrook, G. L. 2006. Synaptic and extrasynaptic NMDA receptor NR2 subunits in cultured hippocampal neurons. J Neurophysiol. 95:1727-1734.

Tigaret, C. M., Thalhammer, A., Rast, G. F., Specht, C. G., Auberson, Y. P., Stewart, M. G., and Schoepfer,R. 2006. Subunit dependencies of NMDA receptor-induced AMPA receptor internalization. Mol. Pharmacol.
Tovar, K. R. and Westbrook, G. L. 1999. The incorporation of NMDA receptors with a distinct subunit composition at nascent hippocampal synapses in vitro. J. Neurosci 19: 4180

Tovar, K. R., and Westbrook, G. L. 2002. Mobile NMDA receptors at hippocampal synapses. Neuron 34:255-264.

Tricklebank et al. 1994. The anticonvulsant and behavioural profile of L-687,414, a partial agonist acting at the glycine modulatory site on the N-methyl-D-aspartate (NMDA) receptor complex. Br. J. Pharmacol. 113(3): 729-36
Wang, Y., Ju, W., Liu, L., Fam, S., D'Souza, S., Taghibiglou, C., Salter, M., and Wang, Y. T. 2004. alpha-Amino-3-hydroxy-5-methylisoxazole-4-propionic acid subtype glutamate receptor (AMPAR) endocytosis is essential for N-methyl-D-aspartate-induced neuronal apoptosis. J. Biol. Chem. 279:41267-41270.
Watson et al. 1990. D-cycloserine acts as a partial agonist at the glycine modulatory site of the NMDA receptor expressed in Xenopus oocytes. Brain Res. 510(1): 158-60
Weitlauf, C., Honse, Y., Auberson, Y. P., Mishina, M., Lovinger, D. M., and Winder, D. G. 2005. Activation of NR2A-containing NMDA receptors is not obligatory for NMDA receptor-dependent long-term potentiation. J Neurosci. 25:8386-8390.
White et al. 2000. In vitro and in vivo characterization of conantokin-R, a selective NMDA receptor antagonist isolated from the venom of the fish-hunting snail Conus radiatus. J. Pharmacol. Exp. Ther. 292(1):425-32
White, H. S., McCabe, R. T., Armstrong, H., Donevan, S. D., Cruz, L. J., Abogadie, F. C., Torres, J., Rivier, J. E., Paarmann, I., Hollmann, M., and Olivera, B. M. Jan. 1, 2000. In Vitro and In Vivo Characterization of Conantokin-R, a Selective Nmda Receptor Antagonist Isolated from the Venom of the Fish-Hunting Snail Conus radiatus1. J. Pharmacol. Exp. Ther. 292(1):425-432
Whittemore, E. R., Ilyin, V. I., and Woodward, R. M. Jul. 1, 1997. Antagonism of N-Methyl-D-Aspartate Receptors by sigma Site Ligands: Potency, Subtype-Selectivity and Mechanisms of Inhibition. J. Pharmacol. Exp. Ther. 282(1):326-338
Williams K, Romano C, Dichter M A, Molinoff P B. 1991. Modulation of the NMDA receptor by polyamines. Life Sci.; 48(6):469-98.
Williams et al. 2002. Selective NR2B NMDA receptor antagonists are protective against staurosporine-induced apoptosis. Eur. J. Pharmacol. 452(1):135-36
Williams et al. 2003. Pharmacology of delta2 glutamate receptors: effects of pentamidine and protons. J. Pharmacol. 305(2):740-48
Wong, T. P., Liu, L., Sheng, M., and Wang, Y. T. 2005. Response to Comment on “Role of NMDA Receptor Subtypes in Governing the Direction of Hippocampal Synaptic Plasticity”. Science 305:1912b.
Xiong, Z. G., Zhu, X. M., Chu, X. P., Minami, M., Hey, J., Wei, W. L., MacDonald, J. F., Wemmie, J. A., Price, M. P., Welsh, M. J. et al 2004. Neuroprotection in ischemia: blocking calcium-permeable acid-sensing ion channels. Cell 118:687-698.
Yaghoubim, N., Malayev, A., Russek, S. J., Gibbs, T. T., and Farb, D. H. Aug. 24, 1998. Neurosteroid modulation of recombinant ionotropic glutamate receptors. Brain Res. 803(1-2):153-60
Yamada et al. 2002. PSD-95 eliminates Src-induced potentiation of NR1/NR2A-subtype NMDA receptor channels and reduces high-affinity zinc inhibition. J. Neurochem. 91(4): 759-64
Yang and Leonard. 2001. Identification of mouse NMDA receptor subunit NR2A C-terminal tyrosine sites phosphorylated with v-Src. J. Neurochem. 77(2): 580-88
Yu, S. P., Canzoniero, L. M., and Choi, D. W. 2001. Curr. Opin. Cell Biol. 13:405-411
Zhou, M., and Baudry, M. 2006. Developmental changes in NMDA neurotoxicity reflect developmental changes in subunit composition of NMDA receptors. J Neurosci. 26:2956-2963.
Zhou, M., and Baudry, M. 2006. Developmental changes in NMDA neurotoxicity reflect developmental changes in subunit composition of NMDA receptors. J Neurosci. 26:2956-2963.
Zhu, Y., Pak, D., Qin, Y., McCormack, S. G., Kim, M. J., Baumgart, J. P., Velamoor, V., Auberson, Y. P., Osten, P., van, A. L. et al 2005. Rap2-JNK removes synaptic AMPA receptors during depotentiation. Neuron 46:905-916.
Zoghbi, H. Y., Gage, F. H., and Choi, D. W. 2000. Neurobiology of disease. Curr. Opin. Neurobiol. 10:655-660.

What is claimed is:

1. A method of modulating N-methyl-D aspartate (NMDA) receptor subtype activity in a neuron having NMDA receptor subunit 2A (NR2A)-containing NMDA receptors and NMDA receptor subunit 2B (NR2B)-containing NMDA receptors, the method comprising treating the neuron with an effective amount of a combination of:
(a) an NR2B-containing NMDA receptor-specific antagonist selected from the group consisting of Ro 25-6981 hydrochloride; Ro 64-1908; Conantokin G; Conantokin R; Felbamate; CP-101 ,606; Ifenprodil; HON0001; Pentamidine isethionate; Ro 8-4304; Eliprodil; (3R,4S)-3-[4-(4-fluorophenyl)-4-hydroxypiperidin-1-yl]chroman-4,7-diol; 1-Benzyloxy-4,5-dihydro-1 H-imidazol-2-yl-amine; CI-1041; Co-101,244; RG-13579; RG-1103; CGX-1007; CR 3394; and, (E)-N-(2-[11 C]methoxybenzyl)-3-phenyl-acrylamidine; and
(b) an NR2A-containing NMDA receptor agonist selected from the group consisting of D-cycloserine; 1-Aminocyclopropanecarboxylic acid and 1-Aminocyclopropanecarboxylic acid hydrochloride; CR 2249; Glycine; D-serine; L-687414; (+)-HA 966; and DL-(tetraziol-5-yl)glycine,
so as to effect a neuroprotective reduction in the effect of excitotoxic NMDA receptor activity in the neuron.

2. A method of neuroprotection in a subject, comprising treating the subject with a therapeutically effective amount of a combination of:
(a) an NR2B-containing NMDA receptor-specific antagonist selected from the group consisting of Ro 25-6981 hydrochloride; Ro 64-1908; Conantokin G; Conantokin R; Felbamate; CP-101 ,606; Ifenprodil; HON0001; Pentamidine isethionate; Ro 8-4304; Eliprodil; (3R,4S)-3-[4-(4-fluorophenyl)-4- hydroxypiperidin-1-yl]chroman-4,7-diol; 1-Benzyloxy-4,5-dihydro-1H-imidazol-2-yl-amine; CI-1041; Co-101,244; RG-13579; RG-1103; CGX-1007; CR 3394; and, (E)-N-(2-[11 C]methoxybenzyl)-3-phenyl-acrylamidine; and
(b) an NR2A-containing NMDA receptor agonist selected from the group consisting of D-cycloserine; 1-Aminocyclopropanecarboxylic acid and 1-Aminocyclopropanecarboxylic acid hydrochloride; CR 2249; Glycine; D-serine; L-687414; (+)-HA 966; and DL-(tetraziol-5-yl)glycine,
so as to effect a neuroprotective reduction in excitotoxic NMDA receptor activity in a neuronal tissue in the subject.

3. The method of claim 2, wherein the subject is a human patient that has a neurodegenerative condition.

4. The method of claim 2, wherein the subject is a human patient that has a condition selected from the group consisting of Alzheimer's disease; Parkinson's disease; amyotrophic lateral sclerosis; Huntington's disease; cognitive impairment associated with schizophrenia; chemotherapy-induced neuropathy; Down's syndrome; Korsakoff's disease; cerebral palsy; epilepsy; neuronal ischemia; neuronal reperfusion injury; neuronal trauma; neuronal hemorrhage; neuronal infection; stroke; and neuronal exposure to a toxic substance.

5. The method of claim 2, wherein the NR2A-containing NMDA receptor agonist is glycine, and wherein said subject is treated for stroke.

6. The method of claim 2, wherein the NR2B-containing NMDA receptor-specific antagonist is Ro 25-6981 hydrochloride.

7. A method of treating a human subject for stroke, comprising treating the subject with a therapeutically effective amount of a combination of:
(a) an NR2B-containing NMDA receptor-specific antagonist selected from the group consisting of Ro 25-6981 hydrochloride; Ro 64-1908; Conantokin G; Conantokin R; Felbamate; CP-101 ,606; Ifenprodil; HON0001; Pentamidine isethionate; Ro 8-4304; Eliprodil; (3R,4S)-3-[4-(4-fluorophenyl)-4-hydroxypiperidin-1-yl]chroman-4,7-diol; 1-Benzyloxy-4,5-dihydro-1H-imidazol-2-yl-amine; CI-1041; Co-101,244; RG-13579; RG-1103; CGX-1007; CR 3394; and, (E)-N-(2-[11 C]methoxybenzyl)-3-phenyl-acrylamidine; and
(b) an NR2A-containing NMDA receptor agonist selected from the group consisting of D-cycloserine; 1-Aminocyclopropanecarboxylic acid and 1-Aminocyclopropanecarboxylic acid hydrochloride; CR 2249; Glycine; D-serine; L-687414; (+)-HA 966; and DL-(tetraziol-5-yl)glycine.

8. The method of claim 7, wherein the NR2B-containing NMDA receptor-specific antagonist is Ro 25-6981 and the NR2A-containing NMDA receptor agonist is glycine.
